SEQUENCE LISTING (80 sequences)

SEQ ID NO: 1 - DNA, 57 bp, synthetic construct
  source 1..57: mol_type = other DNA
cacttttgtcttatacttactagtgccaccatggtttctaaactgagccagctgcag

SEQ ID NO: 2 - DNA, 73 bp, synthetic construct
  source 1..73: mol_type = other DNA
ttccacgtctcctgcttgctttaacagagagaagttcgtggctccggagccctgggaggaagaggccatctgg

SEQ ID NO: 3 - DNA, 68 bp, synthetic construct
  source 1..68: mol_type = other DNA
tatgctctcttgaaattggctggagatgttgagagcaaccctggacctgtcagcgtgaacgcgcccct

SEQ ID NO: 4 - DNA, 59 bp, synthetic construct
  source 1..59: mol_type = other DNA
agaggatccccgggtaccgagctcgaattcctagataacttcctgcttggtgatggtcg

SEQ ID NO: 5 - DNA, 65 bp, synthetic construct
  source 1..65: mol_type = other DNA
tctctgttaaagcaagcaggagacgtggaagaaaaccccggtcctgtcagcgtgaacgcgcccct

SEQ ID NO: 6 - DNA, 77 bp, synthetic construct
  source 1..77: mol_type = other DNA
ctcctccacgtcaccgcatgttagaagacttcctctgccctctccggagccgataacttcctgcttggtgatggtcg

SEQ ID NO: 7 - DNA, 51 bp, synthetic construct
  source 1..51: mol_type = other DNA
cacttttgtcttatacttactagtgccaccatggtcagcgtgaacgcgccc

SEQ ID NO: 8 - DNA, 77 bp, synthetic construct
  source 1..77: mol_type = other DNA
ttccacgtctcctgcttgctttaacagagagaagttcgtggctccggagccgataacttcctgcttggtgatggtcg

SEQ ID NO: 9 - DNA, 65 bp, synthetic construct
  source 1..65: mol_type = other DNA
agtcttctaacatgcggtgacgtggaggagaatcccggccctctgggctcagtgaagatggaggc

SEQ ID NO: 10 - DNA, 82 bp, synthetic construct
  source 1..82: mol_type = other DNA
ctcaacatctccagccaatttcaagagagcataattagtacactgtccggagccggatgcattaagcaaagagcgggaatag

SEQ ID NO: 11 - DNA, 68 bp, synthetic construct
  source 1..68: mol_type = other DNA
tctctgttaaagcaagcaggagacgtggaagaaaaccccggtcctctgggctcagtgaagatggaggc

SEQ ID NO: 12 - DNA, 79 bp, synthetic construct
  source 1..79: mol_type = other DNA
ctcctccacgtcaccgcatgttagaagacttcctctgccctctccggagccggatgcattaagcaaagagcgggaatag

SEQ ID NO: 13 - DNA, 65 bp, synthetic construct; identical to SEQ ID NO: 9
  source 1..65: mol_type = other DNA

SEQ ID NO: 14 - DNA, 61 bp, synthetic construct
  source 1..61: mol_type = other DNA
agaggatccccgggtaccgagctcgaattcctaggatgcattaagcaaagagcgggaatag

SEQ ID NO: 15 - DNA, 68 bp, synthetic construct
  source 1..68: mol_type = other DNA
tctctgttaaagcaagcaggagacgtggaagaaaaccccggtcctaacgcgcagctgaccatggaagc

SEQ ID NO: 16 - DNA, 82 bp, synthetic construct
  source 1..82: mol_type = other DNA
ctcctccacgtcaccgcatgttagaagacttcctctgccctctccggagcctgctttggtacaagtgcttgatgaagaagat

SEQ ID NO: 17 - DNA, 65 bp, synthetic construct
  source 1..65: mol_type = other DNA
agtcttctaacatgcggtgacgtggaggagaatcccggccctaacgcgcagctgaccatggaagc

SEQ ID NO: 18 - DNA, 64 bp, synthetic construct
  source 1..64: mol_type = other DNA
agaggatccccgggtaccgagctcgaattcctatgctttggtacaagtgcttgatgaagaagat

SEQ ID NO: 19 - DNA, 69 bp, synthetic construct
  source 1..69: mol_type = other DNA
tatgctctcttgaaattggctggagatgttgagagcaaccctggacctgacagggccccacagagacag

SEQ ID NO: 20 - DNA, 55 bp, synthetic construct
  source 1..55: mol_type = other DNA
agaggatccccgggtaccgagctcgaattcctacttggacagccccacaggtgac

SEQ ID NO: 21 - DNA, 72 bp, synthetic construct
  source 1..72: mol_type = other DNA
tatgctctcttgaaattggctggagatgttgagagcaaccctggacctaccatgaccctccacaccaaagca

SEQ ID NO: 22 - DNA, 54 bp, synthetic construct
  source 1..54: mol_type = other DNA
agaggatccccgggtaccgagctcgaattcctagaccgtggcagggaaaccctc

SEQ ID NO: 23 - DNA, 71 bp, synthetic construct
  source 1..71: mol_type = other DNA
tatgctctcttgaaattggctggagatgttgagagcaaccctggacctgaagtgcagttagggctgggaag

SEQ ID NO: 24 - DNA, 58 bp, synthetic construct
  source 1..58: mol_type = other DNA
agaggatccccgggtaccgagctcgaattcctactgggtgtggaaatagatgggcttg

SEQ ID NO: 25 - DNA, 20 bp, synthetic construct
  source 1..20: mol_type = other DNA
tgcggcctcttccagaaact

SEQ ID NO: 26 - DNA, 26 bp, synthetic construct
  source 1..26: mol_type = other DNA
cctttggcacaatgaagtgggtaacc

SEQ ID NO: 27 - DNA, 20 bp, synthetic construct
  source 1..20: mol_type = other DNA
agacagcggcagagactatg

SEQ ID NO: 28 - DNA, 18 bp, synthetic construct
  source 1..18: mol_type = other DNA
cagcacttcctgaatgag

SEQ ID NO: 29 - DNA, 23 bp, synthetic construct
  source 1..23: mol_type = other DNA
cattactgacttccgtgctacat

SEQ ID NO: 30 - DNA, 22 bp, synthetic construct
  source 1..22: mol_type = other DNA
gccggcatggagctgtttttat

SEQ ID NO: 31 - DNA, 20 bp, synthetic construct
  source 1..20: mol_type = other DNA
tgtgcctgagaacaccagag

SEQ ID NO: 32 - DNA, 20 bp, synthetic construct
  source 1..20: mol_type = other DNA
gcaagctgaaggacctgtct

SEQ ID NO: 33 - DNA, 20 bp, synthetic construct
  source 1..20: mol_type = other DNA
tgggctcagtgaagatggag

SEQ ID NO: 34 - DNA, 19 bp, synthetic construct
  source 1..19: mol_type = other DNA
gtgtccgtgatcgcagacc

SEQ ID NO: 35 - DNA, 20 bp, synthetic construct
  source 1..20: mol_type = other DNA
gcccttttacatcgaggaca

SEQ ID NO: 36 - DNA, 19 bp, synthetic construct
  source 1..19: mol_type = other DNA
tggccatggacacgtacag

SEQ ID NO: 37 - DNA, 21 bp, synthetic construct
  source 1..21: mol_type = other DNA
catggccaagattgacaacct

SEQ ID NO: 38 - DNA, 22 bp, synthetic construct
  source 1..22: mol_type = other DNA
gtgttgcctctatccttcccat

SEQ ID NO: 39 - DNA, 21 bp, synthetic construct
  source 1..21: mol_type = other DNA
catgagtgtggatccagcttg

SEQ ID NO: 40 - DNA, 21 bp, synthetic construct
  source 1..21: mol_type = other DNA
actgaactcaccccagatcaa

SEQ ID NO: 41 - DNA, 20 bp, synthetic construct
  source 1..20: mol_type = other DNA
ggagccatgtctggtaacgg

SEQ ID NO: 42 - DNA, 20 bp, synthetic construct
  source 1..20: mol_type = other DNA
acacagtgcccatccccaaa

SEQ ID NO: 43 - DNA, 20 bp, synthetic construct
  source 1..20: mol_type = other DNA
agtgagaggcaacctggaga

SEQ ID NO: 44 - DNA, 20 bp, synthetic construct
  source 1..20: mol_type = other DNA
ccctcctcggtgacttatcc

SEQ ID NO: 45 - DNA, 21 bp, synthetic construct
  source 1..21: mol_type = other DNA
gagcctgcatctccaccttat

SEQ ID NO: 46 - DNA, 18 bp, synthetic construct
  source 1..18: mol_type = other DNA
ctacggcagccccagttt

SEQ ID NO: 47 - DNA, 20 bp, synthetic construct
  source 1..20: mol_type = other DNA
ggcgtcctcgtggaagtgac

SEQ ID NO: 48 - DNA, 25 bp, synthetic construct
  source 1..25: mol_type = other DNA
ccaccgccatcttcttcctgcctga

SEQ ID NO: 49 - DNA, 20 bp, synthetic construct
  source 1..20: mol_type = other DNA
cgcacggaatttgaacagta

SEQ ID NO: 50 - DNA, 20 bp, synthetic construct
  source 1..20: mol_type = other DNA
tggagcccgaagaagaggtg

SEQ ID NO: 51 - DNA, 20 bp, synthetic construct
  source 1..20: mol_type = other DNA
tgatcccagtggatggcagc

SEQ ID NO: 52 - DNA, 22 bp, synthetic construct
  source 1..22: mol_type = other DNA
ggagccctgtgatttggagagt

SEQ ID NO: 53 - DNA, 20 bp, synthetic construct
  source 1..20: mol_type = other DNA
taatgtcagccgctccctcg

SEQ ID NO: 54 - DNA, 24 bp, synthetic construct
  source 1..24: mol_type = other DNA
cagcagtcagccatttcaccatag

SEQ ID NO: 55 - DNA, 22 bp, synthetic construct
  source 1..22: mol_type = other DNA
ccagttgtcaaggagctttagg

SEQ ID NO: 56 - DNA, 23 bp, synthetic construct
  source 1..23: mol_type = other DNA
aggtgactgggaggacttgaggc

SEQ ID NO: 57 - DNA, 22 bp, synthetic construct
  source 1..22: mol_type = other DNA
ctcctgcacaaattcgttttcc

SEQ ID NO: 58 - DNA, 21 bp, synthetic construct
  source 1..21: mol_type = other DNA
gccaggccatctgctcttctt

SEQ ID NO: 59 - DNA, 20 bp, synthetic construct
  source 1..20: mol_type = other DNA
gtggtggaaatagtcccgtg

SEQ ID NO: 60 - DNA, 20 bp, synthetic construct
  source 1..20: mol_type = other DNA
aatctgggttgatgcctccg

SEQ ID NO: 61 - DNA, 21 bp, synthetic construct
  source 1..21: mol_type = other DNA
ggggatagggagagcttagag

SEQ ID NO: 62 - DNA, 20 bp, synthetic construct
  source 1..20: mol_type = other DNA
gacgaggttgagccagtctc

SEQ ID NO: 63 - DNA, 20 bp, synthetic construct
  source 1..20: mol_type = other DNA
agggcgaacattgagagcta

SEQ ID NO: 64 - DNA, 20 bp, synthetic construct
  source 1..20: mol_type = other DNA
gctgcttgagggtacttctg

SEQ ID NO: 65 - DNA, 22 bp, synthetic construct
  source 1..22: mol_type = other DNA
ttcccatatgttcctgcatcag

SEQ ID NO: 66 - DNA, 18 bp, synthetic construct
  source 1..18: mol_type = other DNA
cgctccgcttagcagcat

SEQ ID NO: 67 - DNA, 21 bp, synthetic construct
  source 1..21: mol_type = other DNA
cctgaataagcagatccatgg

SEQ ID NO: 68 - DNA, 20 bp, synthetic construct
  source 1..20: mol_type = other DNA
tggttgccatttccgtcaaa

SEQ ID NO: 69 - DNA, 21 bp, synthetic construct
  source 1..21: mol_type = other DNA
ccacgcgaatcactctcatct

SEQ ID NO: 70 - DNA, 20 bp, synthetic construct
  source 1..20: mol_type = other DNA
ggtgcgacctttcatgcacc

SEQ ID NO: 71 - DNA, 20 bp, synthetic construct
  source 1..20: mol_type = other DNA
acactcggaccacatccttc

SEQ ID NO: 72 - DNA, 20 bp, synthetic construct
  source 1..20: mol_type = other DNA
cggtcgcacttgtcatacac

SEQ ID NO: 73 - DNA, 22 bp, synthetic construct
  source 1..22: mol_type = other DNA
agaaacccttgcatccttcaca

SEQ ID NO: 74 - DNA, 19 bp, synthetic construct
  source 1..19: mol_type = other DNA
gctggcatgtctccctgta

SEQ ID NO: 75 - DNA, 20 bp, synthetic construct
  source 1..20: mol_type = other DNA
gccttgcgcatcatggtgtt

SEQ ID NO: 76 - DNA, 23 bp, synthetic construct
  source 1..23: mol_type = other DNA
gagcttcaggggtgcctcctctg

SEQ ID NO: 77 - DNA, 20 bp, synthetic construct
  source 1..20: mol_type = other DNA
ggatcagggacctgtcacac

SEQ ID NO: 78 - DNA, 20 bp, synthetic construct
  source 1..20: mol_type = other DNA
ttcgccttcccgacttggta

SEQ ID NO: 79 - DNA, 20 bp, synthetic construct
  source 1..20: mol_type = other DNA
caacgaggcgtcaggtgcta

SEQ ID NO: 80 - DNA, 20 bp, synthetic construct
  source 1..20: mol_type = other DNA
gaccccggtcactgagaacc

(19) United States
(12) Patent Application Publication
(10) Pub. No.: US 2024/0409899 A1
(43) Pub. Date: Dec. 12, 2024

(54) PRODUCTION OF HEPATOCYTES

(71) Applicant: Cambridge Enterprise Limited (GB/GB), Cambridgeshire (GB)

(72) Inventors: Ludovic Vallier, Cambridgeshire (GB); Rute Alexandra De Costa Tomaz, Cambridgeshire (GB)

(21) Appl. No.: 18/598,289

(22) Filed: Mar. 7, 2024

Related U.S. Application Data
(63) Continuation of application No. PCT/EP2022/075292, filed on Sep. 12, 2022.

(30) Foreign Application Priority Data
Sep. 10, 2021 (GB) 2112937.4

(51) Int. Cl. C12N 5/071; A61K 35/407; C12N 5/00; C12N 15/86; G01N 33/50

(52) U.S. Cl. CPC C12N 5/067; A61K 35/407; C12N 5/0018; C12N 15/86; G01N 33/5067; C12N 2501/60; C12N 2506/45; C12N 2740/15043; G01N 2500/10

(57) ABSTRACT

This invention relates to a method of producing hepatocytes comprising introducing a set of transcription factors consisting of HNF1A; HNF6; FOXA3; RORc and ERα into a population of iPSCs, and culturing the population, such that hepatocytes are produced. Methods of producing hepatocytes, hepatocytes produced by the methods and their uses and applications are provided.

### CROSS-REFERENCE TO RELATED APPLICATIONS

This application is a continuation of PCT/EP2022/075292, filed Sep. 12, 2022, which claims the benefit of and priority to GB 2112937.4, filed on Sep. 10, 2021, the contents of which are incorporated by reference in their entirety.

### FUNDING

The project leading to this application has received funding from the European Research Council (ERC) under the European Union's Horizon 2020 research and innovation programme (grant agreement No 741707).

### FIELD

The present invention relates to methods for producing hepatocytes, hepatocytes produced by these methods and the uses and applications of these hepatocytes.

### SEQUENCE LISTING

The instant application contains a Sequence Listing XML which has been submitted electronically and is hereby incorporated by reference in its entirety. Said XML copy, created on Sep. 8, 2022, is named 008322182-Aug 31.xml, and is 72,469 bytes in size.

### BACKGROUND

Hepatocytes are the main cell type of the liver, comprising 80% its volume and performing a vast array of vital functions including lipid metabolism, storage of macronutrients, secretion of plasma proteins and xenobiotic detoxification (Gordillo et al., 2015; Si-Tayeb et al., 2010; Trefts et al., 2017).

Diseases affecting these functions are life threatening and end stage forms requires liver transplantation. However, only a limited number of patients can benefit from this therapy due to scarcity of donors and the side effects of immunosuppression.

Cell-based therapy using primary hepatocytes has already been found to be an attractive therapeutic alternative to whole organ transplants (Dhawan et al., 2020). However, primary human hepatocytes (PHHs) are in short supply as they can only be obtained from suboptimal livers unsuitable for transplantation. Furthermore, they display a short life, absence of proliferation and rapid loss of functionality in vitro (Mitry et al., 2002). Similarly, the development of new platforms for drug development and toxicology screen is greatly affected by the lack of robust sources of hepatocytes. For all these reasons, alternative sources of hepatocytes are urgently needed.

Producing hepatocytes from human pluripotent stem cells (hPSCs) using directed differentiation protocols has been shown to be an advantageous alternative to PHHs (Palakkan et al., 2017; Szkolnicka & Hay, 2016; Silier et al 2015; Hay et al 2008). These protocols commonly follow key stages of liver development in vitro and allow the production of hepatocyte-like cells (HLCs) which exhibit key hepatic functions including Albumin secretion, lipid metabolism, glycogen storage, and urea cycle activity. However, HLCs systematically present an immature/foetal-like phenotype lacking the full repertoire of functions of mature hepatocytes (Baxter et al., 2015; Grandy et al., 2019; Yiangou et al., 2018). The development of fully functional hepatocytes in vitro is challenging due to the lack of detailed knowledge concerning the molecular mechanisms driving functional maturation in vivo. Indeed, this process occurs progressively during foetal life, but also after birth when it takes nearly 12 months for the liver to become functional. Mimicking this timeline and the associated combination of metabolic changes, exposure to oxygen, nutrition and microbiome constitutes a major challenge for direct differentiation protocols (Chen et al., 2011).

As an alternative, overexpression of transcription factors has been explored as a way to improve the functionality of in vitro generated hepatocytes (Boon et al., 2020; Nakamori et al., 2016; Zhao et al., 2013). Moreover, trans differentiation of somatic cells into liver cells has been achieved by overexpression of liver-enriched transcriptions factors (LETFs) in mouse and human fibroblasts (Rombaut et al., 2021). Importantly, these LETFs comprise the HNF1, HNF3 (FOXA), HNF4 and HNF6 (ONECUT) families all of which play key roles in coordinating liver development (Gordillo et al., 2015; Lau et al., 2018; Schrem et al., 2002). However, direct cell conversion from somatic cell types has a low efficiency/yield due to the strong epigenetic restrictions present in fully differentiated cells. Furthermore, somatic cells have restricted capacity of proliferation which limits large-scale production of hepatocytes without the use of oncogenic manipulation (Du et al., 2014; Huang et al., 2014).

### SUMMARY

The present inventors have developed a process for producing functionally mature hepatocytes (termed FoP-Heps herein) by forward programming human pluripotent cells. This may be useful, for example, in efficient production of functional hepatocytes, for example for use in the modelling of liver disorders; and the development of therapeutics for liver disorders.

A first aspect of the invention provides a method of producing hepatocytes comprising;
(i) providing a population of iPSCs
(ii) introducing a set of transcription factors consisting of HNF1A; HNF6; FOXA3; RORc and ERα into the population of iPSCs, and
(iii) culturing the population, such that hepatocytes are produced.

A second aspect of the invention provides a method of forward programming iPSCs into hepatocytes comprising;
introducing a set of transcription factors consisting of HNF1A; HNF6; FOXA3; RORc and ERα into a population of iPSCs, and
culturing the population, such that hepatocytes are produced.

A third aspect of the invention provides a method of producing hepatocytes comprising;
(i) providing a population of iPSCs
(ii) introducing a set of transcription factors consisting of HNF1A; HNF6; FOXA3; and RORc into the population of iPSCs, and
(iii) culturing the population, such that hepatocytes are produced.

A fourth aspect of the invention provides a method of forward programming iPSCs into hepatocytes comprising;
introducing a set of transcription factors consisting of HNF1A; HNF6; FOXA3; and RORc into a population of iPSCs, and
culturing the population, such that hepatocytes are produced.

A fifth aspect of the invention provides a population of hepatocytes produced by a method according to the first, second, third or fourth aspects.

A sixth aspect of the invention provides a pharmaceutical composition comprising a population of hepatocytes according to the fifth aspect, and a pharmaceutically acceptable excipient.

A seventh aspect of the invention provides a population of hepatocytes according to the fifth aspect for use in a method of treatment of the human or animal body, for example for use in a method of treatment of a liver disorder in an individual.

An eighth aspect of the invention provides a method of treating a liver disorder comprising;
administering a population of hepatocytes according to the fifth aspect to an individual in need thereof.

A ninth aspect of the invention provides the use of a population of hepatocytes according to the fifth aspect in the manufacture of a medicament for use in the treatment of a liver disorder.

A tenth aspect of the invention provides a method of screening for a compound useful in the treatment of a liver disorder comprising;
contacting a population of hepatocytes according to the fifth aspect with a test compound, and;
determining the effect of the test compound on said hepatocytes or the effect of the hepatocytes on the test compound.

In some embodiments, hepatocytes for use in methods of the tenth aspect may have a disease phenotype, such as a liver disorder phenotype.

An eleventh aspect of the invention provides a method of determining the hepatotoxicity of a compound comprising;
contacting isolated hepatocyte cells according to the fifth aspect with a test compound, and;
determining the effect of the test compound on said hepatocytes.

A twelfth aspect of the invention provides a method of identifying a transcription factor that promotes hepatocyte maturation comprising;
determining the expression of a set of transcription factors in primary human hepatocytes (PHHs) and hepatocyte-like cells (HLCs) produced by in vitro directed differentiation,
identifying a transcription factor in the set whose expression is increased in the PHHs relative to the CLCs,
the identified transcription factor being a candidate transcription factor for the promotion of hepatocyte maturation.

A thirteenth aspect of the invention provides a method of identifying a genetic mutation associated with a liver disorder comprising;
providing a test population of hepatocytes of the fifth aspect, wherein the hepatocytes in the test population each comprise a genetic mutation;
comparing the phenotypes of the test population of hepatocytes with a control population of hepatocytes, wherein the control population does not comprise a genetic mutation; and
identifying a hepatocyte in the test population that displays a disease phenotype, such as a liver disorder phenotype,
wherein the genetic mutation in the identified hepatocyte is a candidate genetic mutation associated with a liver disorder.

A fourteenth aspect of the invention provides a method of identifying a genetic mutation associated with a liver disorder comprising;
providing a test population of hepatocytes of the fifth aspect, wherein the hepatocytes in the test population display a disease phenotype, such as a liver disorder phenotype; and
comparing genomic sequence of the test population of hepatocytes with a control population of hepatocytes, wherein the hepatocytes in the control population do not display the disease phenotype; and
identifying one or more genetic mutations in the genomic sequence of the test population relative to the control population,
wherein the identified one or more genetic mutations are candidate genetic mutations associated with a liver disorder.

A fifteenth aspect of the invention provides a method of identifying a gene associated with a liver disorder comprising;
providing a test population of hepatocytes of the fifth aspect, wherein the hepatocytes in the test population display a disease phenotype, such as a liver disorder phenotype; and
comparing the expression of one or more genes in the test population of hepatocytes with the expression of the one or more genes in a control population of hepatocytes,
wherein a difference in the expression of a gene in the test population relative to the control population is indicative that the gene is associated with a liver disorder.

A test population of hepatocytes suitable for use in methods of the thirteenth to the fifteenth aspects may be produced by a method of the first to the fourth aspects from induced pluripotent stem cells (iPSCs) derived from an individual with a liver disorder.

A sixteenth aspect of the invention provides a kit for producing hepatocytes comprising;
(i) a iPSC source cell and an agent that activates or increases the expression or amount of at least three or more transcription factors; and/or
(ii) one or more nucleic acids encoding a set of transcription factors consisting of HNF1A; HNF6; FOXA3; RORc and optionally Erα.

A kit of the sixteenth aspect may be useful for example in methods of the first to the fourth aspects.

Other aspects and embodiments of the invention are described in more detail below.

### BRIEF DESCRIPTION OF THE FIGURES

FIGS. 1A-1J show that forward programming of hPSCs into hepatocytes with 4 and 3 liver-enriched transcription factors (LETFs). (FIG. 1A) Schematic representation of the two sequentially targeted loci. The human ROSA26 was targeted with a constitutively expressed reverse tetracycline transactivator (rtTA). The AAVS1 locus was targeted with the 4 LETFs HNF1A, HNF6, FOXA3 and HNF4A downstream of a Tet-responsive element (TET). (FIG. 1B) mRNA induction levels of the four factors in targeted hESCs (Targ) relative to untargeted (Untarg) hESCs, stimulated with dox for 24 h (n=3). Data is shown relative to the untargeted control. (FIG. 1C) Immunofluorescence staining of the 4 LETFs in targeted and untargeted hESCs after 24 h of inducible overexpression (iOX) with dox confirming transgene induction. Nuclei were counterstained with DAPI (blue). Scale bar, 200 μm. (FIG. 1D) Schematic representation of the iOX culture conditions for forward programming. Phase contrast images of hESCs targeted with the 4 LETFs after 10 and 15 days of forward programming. (FIG. 1E) mRNA levels of hepatocyte markers (ALB, SERPINA1 and AFP) in hESCs targeted with the 4 LETFs after 10 and 15 days of forward programming. Untargeted hESCs treated with the same protocol as in (FIG. 1D) were used as control (n=4). Statistical difference was calculated with unpaired t-test against untargeted and p-values are displayed for each comparison. (FIG. 1F) CYP3A4 activity levels normalised per cell number (millions) in untargeted and targeted hESCs with the 4 LETFs after 15 days of forward programming (n=5) and HLCs generated by direct differentiation (n=6). Statistical difference between targeted and untargeted cells was calculated with unpaired t-test. (FIG. 1G, FIGS. 1H and 1I) mRNA levels of hepatocyte markers (ALB, SERPINA1 and AFP) in hESCs targeted with the 4 LETFs and with combinations of 3 LETFs (n=4). The factor removed from each construct is indicated. Expression levels were determined after 10, 15, 20 and 25 days of forward programming. Statistical differences were calculated with one-way ANOVA corrected for multiple comparisons compared to 4 LETFs. Significant p-values are shown at each timepoint. All mRNA levels were normalised to the average of 2 housekeeping genes (PBGD and RPLP0). (FIG. 1J) CYP3A4 activity levels normalised per cell number (millions) in hESCs targeted with the 4 LETFs and combinations of 3 LETFs after 10, 15, 20 and 25 days of forward programming (n=3-5). Statistical differences were calculated with one-way ANOVA, corrected for multiple comparisons compared to 4 LETFs. Significant p-values are shown at each timepoint. In all plots, bars represent mean with SD, and individual datapoints are shown for all biological replicates.

FIGS. 2A-2G shows HLCs and PHHs display transcriptomic differences associated with their state of maturation. (FIG. 2A) Immunofluorescence staining of Albumin (yellow) and HNF4A (red) in HLCs differentiated for 30 days. Nuclei were counterstained with DAPI (blue). Scale bar, 100 μm. (FIG. 1B) CYP3A4 activity levels normalised per cell number (millions) in HLCs differentiated for 30 days (n=6) and PHHs (n=4). Bars represent mean with SD, and individual datapoints represent the different biological replicates. Statistical difference was calculated with unpaired t-test. (FIG. 2C) PCA of undifferentiated hiPSCs, HLCs derived from hESC (hESC_HLCs) and hiPSC (hiPSC_HLCs), freshly harvested PHHs (fPHHs) or plated PHHs (pPHHs). (FIG. 2D) Heatmap showing the proportion of genes differentially expressed in each cell type (cluster 1—PHHs, cluster 2—HLCs, cluster 4—hiPSCs) as well as in Heps (HLCs and PHHs) against undifferentiated hiPSCs (cluster 3). (FIGS. 2E and 2F) Dotplot showing the top 15 hits on gene ontology enrichment analysis on genes associated to cluster 1 and cluster 3 as shown in (FIG. 2D). The size of each dot represents number of genes associated to each term and the colours represents the adjusted p-value. (FIG. 2G) Heatmap showing the differential gene expression of transcription factors between PHHs (fresh or plated) and HLCs (hESC and hiPSC derived). (FIG. 2H) Reactome pathway enrichment analysis on transcription factors identified in (FIG. 2G). Differential gene expression was calculated with log 2(fold change) higher than 2 and adjusted p-value <0.05. Hierarchical clustering on samples was generated by Euclidean distance.

FIGS. 3A-3D shows the epigenetic status of regulatory regions differs between states of maturation in HLCs and PHHs. (FIG. 3A) PCA of the global enrichment profile of H3K27ac, H3K4me1 and H3K27me3 across 2 replicates of undifferentiated hiPSCs, hESC and hiPSC-derived HLCs, and PHHs. Average scores were computed for genomic regions of 1000 bp for the entire genome. (FIG. 3B) Average density plots and heatmaps showing enrichment levels for H3K27ac, H3K4me1 and H3K27me3 within a 10 Kb window centred at H3K27ac PHH-unique (blue) or HLC-unique (green) regions. Scales are adjusted to maximum peak intensity for each dataset. (FIG. 3C) Enrichment profiles of H3K27ac, H3K4me1 and H3K27me3 across the UGT1A locus. Profiles are shown for one replicate of undifferentiated hiPSCs, hESC and hiPSC-derived HLCs, and PHHs. Red bars represent PHH-unique H3K27ac peaks. (FIG. 3D) Nuclear receptor motifs identified as overrepresented binding sites at H3K27ac PHH-unique regions.

FIGS. 4A-4G shows forward programming of hESCs into hepatocytes with nuclear receptors. (FIG. 4A) Phase contrast images and (FIG. 4B) immunofluorescence staining for Albumin (yellow) and (FIG. 4C) A1AT (green) in hESCs forward programmed for 20 days with 3TFs alone or in combination with the nuclear receptors RORc, ER a and AR. Nuclei were counterstained with DAPI (blue). Scale bars, 200 μm. (FIG. 4D) mRNA levels of hepatocyte markers (ALB, SERPINA1 and AFP) in FoP-Heps generated with 3TFs alone or in combination with nuclear receptors for 20 and 30 days (n=4). Expression data was normalised to the average of 2 housekeeping genes (PBGD and RPLP0). (FIG. 4E) Protein secretion levels of Albumin, A1AT and AFP in hESC derived FoP-Heps generated with 3TFs alone or in combination with nuclear receptors for 20 days (n=4). Data was normalised per total cell number (millions). (FIG. 4F) CYP3A4 activity levels normalised per cell number (millions) in FoP-Heps targeted with 3TFs with or without nuclear receptors, after 20 and 30 days of forward programming (n=3-6). Statistical differences were calculated with one-way ANOVA, corrected for multiple comparisons compared to 3TFs (day 20). Significant p-values are shown. (FIG. 4G) CYP3A4 fold induction levels in FoP-Heps treated with 100 nM of the ligands as indicated from day 2. Data is normalised to untreated control at day 20 of forward programming (n=3). Significant p-values are shown for paired t-test. In all plots, bars represent mean with SD, and individual datapoints are shown for all biological replicates.

FIGS. 5A-5F shows forward programming of hiPSCs into hepatocytes with 4TFs. (FIG. 5A) Phase contrast images and (FIG. 5B) immunofluorescence staining for Albumin (yellow) and (FIG. 5C) A1AT (green) in hiPSCs forward programmed for 20 days with 3TFs alone or with RORc. Nuclei were counterstained with DAPI (blue). Scale bars, 200 μm. (FIG. 5D) mRNA levels of hepatocyte markers (ALB, SERPINA1 and AFP) in hiPSC derived FoP-Heps generated with 3TFs alone or with RORc for 20 and 30 days (n=4). Statistical differences were calculated with unpaired t-test and significant p-values are shown. All expression data was normalised to the average of 2 housekeeping genes (PBGD and RPLP0). (FIG. 5E) Protein secretion levels of Albumin, A1AT and AFP in hiPSC derived FoP-Heps generated with 3TFs alone or with RORc for 20 days (n=4). Data was normalised per total cell number (millions). (FIG. 5F) CYP3A4 activity levels normalised per cell number (millions) in hiPSC FoP-Heps targeted with 3TFs with or without RORc after 20 days of forward programming (n=6). Statistical difference were calculated with unpaired t-test. In all plots, bars represent mean with SD, and individual datapoints are shown for all biological replicates.

FIGS. 6A-6H shows that RORc promotes functionality of 4TF FoP-Heps. (FIG. 6A) Comparison of CYP3A4 activity levels in FoP-Heps (n=6) against direct differentiation HLCs (n=6) and PHHs (n=4). hESC (eFoP) and hiPSC (iFoP) derived FoP-Heps were targeted with the 4TFs (HNF1A, FOXA3, HNF6 and RORy) and forward programmed for 20 days. Statistical difference between HLCs and FoP-Heps were calculated with unpaired t-test. (FIG. 6B) mRNA levels of phase I (CYP2A6 and CYP2C8) and phase II (UGT1A6) biotransformation enzymes in 4TF FoP-Heps, HLCs and PHHs (n=4). (FIG. 6C) mRNA level of gluconeogenesis (G6PC and PCK1), lipid (PPARα, PPARγ) metabolism, and the nuclear receptor RORy in FoP-Heps, HLCs and PHHs (n=4). Statistical difference between HLCs and the other cell types were calculated with unpaired t-test. (FIG. 6D) Immunofluorescence staining for LDL in FoP-Heps at day 20 of forward programming. Scale bars, 200 μm. Nuclei were counterstained with DAPI (blue). (FIG. 6E) Comparison of mRNA levels of SERPINA1 and UGT1A6 in FoP-Heps cultured in 2D and 3D for up to 20 or 30 days of forward programming (n=4). Statistical difference between 2D and 3D were calculated with unpaired t-test. All expression data was normalised to the average of 2 housekeeping genes (PBGD and RPLP0). (FIG. 6F) BODIPY staining of FoP-Heps cultured in 3D from day 20 of forward programming and treated with fatty acids (oleic acid [OA], palmitic acid [PA] or BSA [Ctr]) as indicated for 7 days. Scale bars, 200 μm. Nuclei were counterstained with DAPI (blue). (FIG. 6G) Cell viability in FoP-Heps treated with the fatty acids as indicated, normalised against FoP-Heps treated with BSA as control (n=4). (FIG. 6H) Cell viability in FoP-Heps treated with 25 mM of acetaminophen (APAP) for 48 h in 3D cultures, normalised against untreated FoP-Heps (n=4). Significant differences were determined with paired t-test. P-values are indicated as *p<0.05, **p<0.01, ***p<0.001, ****p<0.0001. In all plots, bars represent mean with SD, an individual datapoints are shown for all biological replicates.

FIGS. 7A-7G shows ERα promotes functionality of 5TF FoP-Heps. (FIG. 7A) Schematic representation of the combinations of factors cloned into the AAVS1 locus. (FIG. 7B) Phase contrast images in hiPSCs forward programmed for 20 days with 5TFs alone or with estrogen (E2). (FIG. 7C) mRNA levels of hepatocyte markers (ALB, SERPINA1 and AFP) in hiPSC derived FoP-Heps generated with 4TFs, 5TFs, or 5TFs+E2 for 20 days (n=4). Statistical differences were calculated with unpaired t-test and significant p-values are shown. All expression data was normalised to the average of 2 housekeeping genes (PBGD and RPLP0). (FIG. 7D) mRNA levels of phase I (CYP2A6 and CYP2C8) and phase II (UGT1A6) biotransformation enzymes and gluconeogenesis enzymes (G6PC and PCK1), in 4TF, 5TF and 5TF+E2 FoP-Heps (n=4). (FIG. 7E) Comparison of CYP3A4 activity levels in FoP-Heps generated with 4TFs, 5TFs, or 5TFs+E2 and forward programmed for 20 days. (FIG. 7F) BODIPY staining of FoP-Heps generated with 5TFs cultured in 3D from day 20 of forward programming and treated with fatty acids (oleic acid [OA], palmitic acid [PA] or BSA [Ctr]) as indicated for 7 days. Scale bars, 200 μm. Nuclei were counterstained with DAPI (blue). (FIG. 7G) Cell viability in FoP-Heps generated with 5TFs and treated with the fatty acids as indicated, normalised against FoP-Heps treated with BSA as control (n=4).

### DETAILED DESCRIPTION

This invention relates to the forward programming of pluripotent cells into hepatocytes. A set of transcription factors consisting of HNF1A; HNF6; FOXA3; RORc and optionally ERα is introduced into the pluripotent cells. The pluripotent cells are then cultured. The set of transcription factors introduced into the cells imposes a mature hepatocyte phenotype on the pluripotent cells i.e. the population of pluripotent cells is forward programmed by the set of transcription factors into functionally mature hepatocytes. These hepatocytes may be useful, for example, in disease modelling, drug screening and therapeutic methods.

Forward programming is the direct imposition of a more differentiated phenotype on a pluripotent stem cell or other precursor cell which bypasses normal differentiation pathway; i.e. the cell does not pass progressively through intermediate stages of differentiation. For example, a pluripotent stem cell (PSC) which is forward programmed into a hepatocyte does not differentiate sequentially through endoderm, foregut, hepatoblast and foetal hepatocyte stages before displaying the mature hepatocyte phenotype. Forward programming by direct overexpression of transcription factors in PSCs has been successfully used to generate neurons, skeletal myocytes, and oligodendrocytes (Pawlowski et al., 2017).

Pluripotent stem cells (PSCs) are capable of self-renewal in vitro, exhibit an undifferentiated phenotype and are potentially capable of differentiating into any foetal or adult cell type of any of the three germ layers (endoderm, mesoderm and endoderm). A pluripotent stem cell is distinct from a totipotent stem cell and cannot give rise to extraembryonic cell lineages. The population of PSCs may be clonal i.e. genetically identical cells descended from a single common ancestor cell. PSCs may express one or more of the following pluripotency associated markers: Oct4, Sox2, Alkaline Phosphatase, POU5f1, SSEA-3, Nanog, SSEA-4, Tra-1-60, KLF-4 and c-myc, preferably one or more of POU5f1, NANOG and SOX2. A PSC may lack markers associated with specific differentiative fates, such as Bra, Sox17, FoxA2, αFP, Sox1, NCAM, GATA6, GATA4, Hand1 and CDX2. In particular, a PSC may lack markers associated with endodermal fates.

Preferably, the PSCs are human PSCs (hPSCs).

PSCs may include embryonic stem cells (ESCs) and non-embryonic stem cells, for example foetal stem cells, adult stem cells, amniotic stem cells, cord stem cells and induced pluripotent stem cells (iPSCs). In some embodiments, the PSCs are not human embryonic stem cells. In some embodiments, the PSCs are not human embryonic cells. Suitable techniques for generating PSCs are well-known in the art.

Preferably, the PSCs are iPSCs, more preferably human iPSCs (hiPSCs).

iPSCs are pluripotent cells which are derived from non-pluripotent, fully differentiated ancestor or antecedent cells. Suitable ancestor cells include somatic cells, such as adult fibroblasts and peripheral blood cells. Ancestor cells are typically reprogrammed by the introduction of pluripotency genes or proteins, such as Oct4, Sox2 and Sox1 into the cell. The genes or proteins may be introduced into the differentiated cells by any suitable technique, including plasmid or more preferably, viral transfection or direct protein delivery. Other genes, for example Klf genes, such as Klf-1, -2, -4 and -5; Myc genes such as C-myc, L-myc and N-myc; nanog; and Lin28 may also be introduced into the cell to increase induction efficiency. Following introduction of the pluripotency genes or proteins, the ancestor cells may be cultured. Cells expressing pluripotency markers may be isolated and/or purified to produce a population of iPSCs. Techniques for the production of iPSCs are well-known in the art (Yamanaka et al Nature 2007; 448:313-7; Yamanaka 6 2007 Jun. 7; 1(1):39-49; Kim et al Nature. 2008 Jul. 31; 454(7204):646-50; Takahashi Cell. 2007 Nov. 30; 131(5):861-72. Park et al Nature. 2008 Jan. 10; 451(7175):141-6; Kimet et al Cell Stem Cell. 2009 Jun. 5; 4(6):472-6; Vallier, L., et al. Stem Cells, 2009. 9999(999A): p. N/A). iPSCs for use in the present methods may be derived from somatic cells, such as fibroblasts or blood cells, which have a normal (i.e. non-disease associated) genotype, for example cells obtained from an individual with a normal genetic background e.g. an individual without a genetic disorder. The iPSCs may be used to produce hepatocytes with a normal (i.e. non-disease associated) genotype, for example for use in therapy, modelling, screening or other applications.

iPSCs for use in some embodiments of the present methods may be derived from somatic cells or other antecedent cells obtained from an individual with a distinct genetic background. For example, iPSCs may be produced from cells from an individual having a disease condition, an individual having a high risk of a disease condition and/or an individual with a low risk of a disease condition. Disease conditions may include liver disorders e.g. a hepatopathy or other disorder associated with the liver. iPSCs produced from cells obtained from an individual with a distinct genetic background may be used to produce hepatocytes which possess the genetic background, which may be useful in studying the mechanisms of disease conditions, such as liver disorders, and in identifying therapeutic targets.

Conventional techniques may be employed for the culture and maintenance of PSCs (Vallier, L. et al Dev. Biol. 275, 403-421 (2004), Cowan, C. A. et al. N. Engl. J. Med. 350, 1353-1356 (2004), Joannides, A. et al. Stem Cells 24, 230-235 (2006) Klimanskaya, I. et al. Lancet 365, 1636-1641 (2005), Ludwig, T. E. et al. Nat. Biotechnol. 24, 185-187 (2006)). PSCs for use in the present methods may be grown in defined conditions or on feeder cells. For example, PSCs may be conventionally cultured in a culture dish on a layer of feeder cells, such as irradiated mouse embryonic fibroblasts (MEF), at an appropriate density (e.g. 10⁵ to 10⁶ cells/60 mm dish), or on an appropriate substrate with feeder conditioned or defined medium. Pluripotent cells for use in the present methods may be passaged by enzymatic or mechanical means.

In preferred embodiments, PSCs for use in the present methods may be cultured in a culture medium that is chemically defined.

A chemically defined medium is a nutritive solution for culturing cells which contains only specified components, preferably components of known chemical structure. A chemically defined medium is devoid of undefined components or constituents which include undefined components, such as feeder cells, stromal cells, serum, serum albumin and complex extracellular matrices, such as Matrigel™. In some embodiments, the chemically defined medium is humanised. A humanised chemically defined medium is devoid of components or supplements derived or isolated from non-human animals, such as Foetal Bovine Serum (FBS) and Bovine Serum Albumin (BSA), and mouse or other feeder cells. Proteins in a humanised CDM may be recombinant human proteins. Conditioned medium includes undefined components from cultured cells and is not chemically defined. Suitable chemically defined media are well known in the art and described in more detail below. Media and ingredients thereof may be obtained from commercial sources (e.g. Gibco, Roche, Sigma, Europabioproducts, Cellgenix, Life Sciences).

In some embodiments, a chemically defined medium may comprise a chemically defined basal medium supplemented with a serum-free media supplement and/or one or more additional components, for example transferrin, 1-thioglycerol, defined lipids, L-glutamine or substitutes, such as GlutaMAX-1™ nicotinamide, dexamethasone, selenium, pyruvate, buffers, such as HEPES, sodium bicarbonate, glucose and antibiotics such as penicillin and streptomycin and optionally polyvinyl alcohol; polyvinyl alcohol and insulin; serum albumin; or serum albumin and insulin. Suitable chemically defined basal medium, such as Advanced Dulbecco's modified eagle medium (DMEM) (Price et al Focus (2003) 25 3-6), Iscove's Modified Dulbecco's medium (IMDM), William's E medium and RPMI-1640 (Moore, G. E. and Woods L. K., (1976) Tissue Culture Association Manual. 3, 503-508; see Table 3) are known in the art and available from commercial sources (e.g. Sigma-Aldrich MI USA; Life Technologies USA). Other suitable chemically defined basal medium are known in the art and available from commercial sources (e.g. Sigma-Aldrich MI USA; Life Technologies USA). Suitable serum-free media supplements include B27 (Brewer et al Brain Res (1989) 494 65-74; Brewer et al J. Neurosci Res 35 567-576 (1993); Brewer et al Focus 16 1 6-9; Brewer et al (1995) J. Neurosci. Res. 42:674-683; Roth et al J Trace Elem Med Biol (2010) 24 130-137) and NS21 (Chen et al J. Neurosci Meths (2008) 171 239-247). Serum-free media supplements, such as B27 and N21, are well-known in the art and widely available commercially (e.g. Invitrogen; Sigma Aldrich Inc).

Suitable chemically defined media for use in culturing PSCs include E8 medium, which comprises DMEM/F12 supplemented with insulin, selenium, transferrin, L-ascorbic acid, FGF2, and TGFβ (or NODAL or Activin) and pH adjusted with NaHCO₃(Chen et al 2011 Nat Methods 8 (5) 424-U76); and E6 medium, which comprises DMEM/F12 supplemented with insulin, for example at 0.5 μg/ml to 70 μg/ml, transferrin, for example at a concentration of 1.5 μg/ml to 150 μg/ml, L-ascorbic acid, example at 30 μg/ml to 120 μg/ml, FGF2 and pH adjusted with NaHCO₃(Chen et al 2011 Nat Methods 8 (5) 424-U76).

Other suitable chemically defined media include CDM-PVA (Johansson and Wiles (1995) Mol Cell Biol 15, 141-151), which comprises a basal medium supplemented with polyvinyl alcohol, insulin, transferrin and defined lipids. For example, a CDM-PVA medium may consist of: 50% Iscove's Modified Dulbecco's Medium (IMDM) plus 50% Ham's F12 with GlutaMAX-1™ or 50% F12 NUT-MIX (Gibco, supplemented with 1% chemically defined lipid concentrate, 450 μM 1-thiolglycerol, 15 μg/ml transferrin, 1 mg/ml polyvinyl alcohol, 7 μg/ml Insulin. Other suitable chemically defined nutrient media include hESC maintenance medium (CDMA) which is identical to the CDM-PVA described above with the replacement of PVA with 5 mg/ml BSA; and RPMI basal medium supplemented with B27 and Activin (for example at least 50 ng/ml). CDM-PVA media are described in Vallier et al 2009 PLoS ONE 4: e6082. doi: 10.1371; Vallier et al 2009 Stem Cells 27: 2655-2666, Touboul 2010 51: 1754-1765. Teo et al 2011 Genes & Dev. (2011) 25: 238-250 and Peterson & Loring Human Stem Cell Manual: A Laboratory Guide (2012) Academic Press.

A population of PSCs may be cultured in the methods described herein in a plating medium for 12 to 36 hours, preferably about 24 hours. Suitable plating media include E8 medium. The plating medium may be supplemented with a Rho-associated, coiled-coil containing protein kinase (ROCK) inhibitor, for example 1 to 100 μM ROCK inhibitor, such as 10 μM Y-27632.

PSCs are forward programmed to become hepatocytes in the methods described herein through the introduction of a set of transcription factors into the PSCs. This introduction increases the intracellular levels of the set of transcription factors in the PSCs and elicits the conversion of the PSCs in the population into hepatocytes.

Transcription factors are DNA binding proteins which regulate the expression of genes in cells. Preferably, the transcription factors introduced into the PSCs are human transcription factors. The set of transcription factors used in the methods described herein to forward program PSCs into hepatocytes consists of HNF1A; HNF6; FOXA3; RORc and optionally ERα. For example, the set of transcription factors may consist of HNF1A; HNF6; FOXA3; and RORc; or the set of transcription factors may consist of HNF1A; HNF6; FOXA3; RORc and ERα.

Hepatocyte nuclear factor 1 homeobox A (HNF1A; Gene ID No: 6927) is a liver enriched transcription factor (LETF). HNF1A may have the reference amino acid sequence of NP_00536.6 or NP_001293108.2 and may be encoded by the reference nucleotide amino acid sequence of NM_00545.8 or NM_001306179.2.

Hepatocyte nuclear factor (HNF6; Gene ID 3175; also called one cut homeobox 1; ONECUT1)) is a liver enriched transcription factor (LETF). HNF6 may have the reference amino acid sequence of NP_004489.1 and may be encoded by the reference nucleotide amino acid sequence of NM_004498.4.

Forkhead box A3 (FOXA3; Gene ID: 3171; also called hepatocyte nuclear factor 3-gamma HNF3G) is a liver enriched transcription factor (LETF). FOXA3 may have the reference amino acid sequence of NP_004488.2 and may be encoded by the reference nucleotide amino acid sequence of NM_004497.3.

RAR related orphan receptor C (RORc; Gene ID: 6097) is a nuclear transcription factor expressed mainly in immune cells. RORc may have the reference amino acid sequence of NP_001001523.1 or NP_005051.2 and may be encoded by the reference nucleotide amino acid sequence of NM_001001523.2 or NM_005060.4.

Estrogen receptor alpha (ESR1, Era, ERα or NR3A1; Gene ID: 2099) is a nuclear receptor activated by estrogen. ERα may have the reference amino acid sequence of NP_000116.2 or NP_001116212.1 and may be encoded by the reference nucleotide amino acid sequence of NM_000125.4 or NM_001122740.2

Suitable transcription factors for use as described herein may comprise the reference database amino sequence or a variant thereof. A suitable variant may have at least 50%, at least 60%, at least 70%, at least 80%, at least 90%, at least 95%, or at least 98% sequence identity to the reference sequence. Amino acid sequence identity is generally defined with reference to the algorithm GAP (GCG Wisconsin Package™, Accelrys, San Diego CA). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST or TBLASTN (which use the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), generally employing default parameters. Particular sequence variants may differ from a reference sequence by insertion, addition, substitution or deletion of 1 amino acid, 2, 3, 4, 5-10, 10-20 or 20-30 amino acids.

Suitable transcription factor nucleic acids and proteins may be produced using routine recombinant techniques or obtained from commercial suppliers (e.g. R&D Systems, Minneapolis, MN, USA; Cellgenix, DE; Life Technologies, USA).

In some preferred embodiments, the defined set of transcription factors are the only transcription factors introduced into the PSCs. Other transcription factors, such as HNF4A, are not introduced into the PSCs.

In other embodiments, one or more additional transcription factors selected from the group consisting of NR1, CUX2, AR, ZNF558, TSHZ2, TBX15, NF1X, NF1B, ATOH8, ZMAT1, ONECUT2, ZNF3858, FOS, FOSB, NR113, NPAS2, L3MBTL4, JAZF1, NF1A, ZNF680, HNF4G, CREBL2, DMRTA1, IRF6, ARIDSA, SOX5, ZBTB20, ZNF704, ZEB1, ZNF367, NR1H4, KLF15, HLF and NR4A2 may also be introduced into the PSCs in addition to the set of transcription factors.

The set of transcription factors may be introduced into the PSCs in the form of nucleic acids (Warren L et al. Cell Stem Cell. 2010 Nov. 5; 7(5):618-30) or proteins (Zhou H, et al Cell Stem Cell. 2009 May 8; 4(5):381-4).

Following introduction of the reprogramming nucleic acids or proteins, the population of treated cells may be cultured.

In some embodiments, the set of transcription factors may be introduced into the PSCs by expressing heterologous nucleic acid encoding the set of transcription factors in the PSCs. The amount of the transcription factors in the set is thereby increased in the PSCs. The amount of transcription factors in the set may be increased by at least 10%, 20%, 30%, 40%, 50%, 60%, 70%, 80%, 90%, or more relative to a control (e.g., a PSC into which the set of transcription factors has not been introduced).

The nucleic acid may be operably linked to inducible or non-inducible regulatory elements within a suitable vector, for example a plasmid or viral vector, such as a retroviral or lentiviral vector, for expression within the cells. Vectors containing the nucleic acid are then transfected into the PSCs. Any convenient technique for the transfection may be employed. In some embodiments, the set of transcription factors may be introduced into the PSCs by a method comprising (a) inserting a nucleic acid encoding a transcriptional regulator protein into a first genetic safe harbour site of a PSC; and (b) inserting one or more nucleic acids encoding a set of transcription factors consisting of: set of transcription factors consisting of HNF1A; HNF6; FOXA3; RORc and optionally ERα into a second genetic safe harbour site of the PSC, said one or more nucleic acids being operably linked to an inducible promoter regulated by the transcriptional regulator protein; and (c) culturing the PSCs, to produce the hepatocytes. Safe harbour loci are well known in the art and include the hROSA26 locus and the AAVS1 locus.

Following transfection, the set of transcription factors is expressed in the PSCs and programs the PSCs to become hepatocytes. The transcription factors may be overexpressed in the PSCs. For example, the set of transcription factors may be expressed at a level that is higher than the endogenous expression level of the of the set of transcription factors in the cells, for example at least 2 fold higher, at least 3 fold higher, at least 2 fold higher or at least 5 fold higher. In some embodiments, transposon-mediated or other random integration transgenesis techniques may be employed. Reprogramming cells through expression of nucleic acid encoding one or more transcription factors is well-known in the art (Takahashi et al 2007; Takahashi et al 2007; Seki et al 2010; Loh et al 2010; Staerk et al 2010).

The expression of the transcription factors from encoding nucleic acid in the iPSCs may be inducible. For example, the encoding nucleic acid may be operably linked to one or more inducible regulatory elements within a suitable vector. Inducible regulatory elements may include tetracycline (Tc) or doxycycline (dox) inducible regulatory elements. Suitable methods for the inducible expression of transcription factors in PSCs are established in the art (see for example Pawlowski et al (2017); WO2018096343A1).

In some preferred embodiments, the PSCs may be programmed to become hepatocytes with minimal or no genetic modification to the cells. Suitable techniques are known in the art and include the use of excisable lentiviral and transposon vectors; repeated application of transient plasmid, episomal and adenovirus or adeno-associated vectors or; the use of small molecules, synthetic mRNA and/or microRNAs (Sidhu K S. Expert Opin Biol Ther. (2011) May; 11(5):569-79; Woltjen K et al (2009) Nature 458 (7239):766-70; Chou B K et al. Cell Res. 2011 21(3):518-29).

In other embodiments, the set of transcription factors may be introduced into the PSCs by contacting transcription factor proteins or transcription factor nucleic acids, such as mRNAs encoding transcription factors, with the population of PSCs. Programming cells though the direct delivery of transcription factor nucleic acids (Warren L et al. Cell Stem Cell. 2010 Nov. 5; 7(5):618-30) or proteins (Zhou H, et al Cell Stem Cell. 2009 May 8; 4(5):381-4) is well-known in the art and any suitable technique may be employed. For example, the combination of transcription factor proteins or nucleic acids may be cultured in the presence of the PSCs under conditions which allow for entry of the proteins or nucleic acid into the cell. In some embodiments, entry of transcription factor proteins into the cell may be facilitated by a membrane penetrating peptide, which may be linked or attached to the transcription factor proteins. The combination of transcription factor proteins or nucleic acids may be introduced into the PSCs by traditional methods such as lipofection, electroporation, calcium phosphate precipitation, particle bombardment and/or microinjection, or may be delivered into cells by a protein delivery agent. For example, the combination of transcription factor proteins or nucleic acids can be introduced into cells by covalently or non-covalently attached lipids, e.g. a myristoyl group.

Transcription factor nucleic acids delivered directly into PSCs may be translatable by endogenous translation factors within the cell. Suitable synthetic mRNAs may be modified. For example, 5-methylcytidine may be substituted for cytidine, and pseudouridine for uridine, followed by phosphatase treatment to produce the transcription factor nucleic acids (Zhou H, et al 2009).

In other embodiments, the set of transcription factors may be introduced into the PSCs by activating expression of endogenous nucleic acid sequences encoding the transcription factors in the population of PSCs. Suitable techniques for endogenous gene activation include Zinc Finger or Transcription like Activator (TAL) techniques and are well established in the art (see for example Hum Gene Ther. 2012 May 15; Zhang P et al. Hum Gene Ther. 2012 November; 23(11):1186-99).

The PSCs may be cultured in a programming medium when the set of transcription factors is introduced.

Preferably, the programming medium is a chemically defined medium. Suitable programming media may comprise a basal culture medium, such as DMEM/F12, supplemented with insulin, for example at 0.5 μg/ml to 70 μg/ml, transferrin, for example at a concentration of 1.5 μg/ml to 150 μg/ml, L-ascorbic acid, example at 30 μg/ml to 120 μg/ml, FGF2, example at 0.05 μg/ml to 0.2 μg/ml, and TGFβ (or NODAL) example at 0.05 μg/ml to 0.2 μg/ml. For example, a suitable chemically defined programming medium may comprise a basal culture medium, such as DMEM/F12, supplemented with insulin, for example at 0.5 μg/ml to 70 μg/ml, transferrin, for example at a concentration of 1.5 μg/ml to 150 μg/ml, and L-ascorbic acid. In some preferred embodiments, the programming medium is E6 medium. E6 medium is described in detail above.

In embodiments in which nucleic acid encoding the set of transcription factors is operably linked to one or more inducible regulatory elements, the programming medium may be supplemented with one or more agents that induce expression of the set of transcription factors. For example, when the encoding nucleic acid is operably linked to one or more doxycycline inducible regulatory elements, the programming medium may be supplemented with doxycycline.

Following the introduction of the set of transcription factors into the PSCs, the cells may be cultured in the programming medium for 12 hours or more, 1 day or more, 2 days or more or 3 days or more, preferably about 1 day.

Methods for culturing mammalian cells, such as PSCs, are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52, Handbook of Stem Cells (ed. R. Lanza) ISBN: 0124366430). Media and ingredients thereof may be obtained from commercial sources (e.g. Gibco, Roche, Sigma, Europa bioproducts, R&D Systems). Standard mammalian cell culture conditions may be employed, for example 37° C., 21% Oxygen, 5% Carbon Dioxide. Culture medium is preferably changed every two days and cells allowed to settle by gravity.

Following culture in the programming medium, the cells may then be cultured in a hepatocyte medium.

A hepatocyte medium is a culture medium that supports the maintenance of hepatocyte phenotype in cultured cells. The hepatocyte medium is preferably a chemically defined medium. Suitable media include Hepatozyme (ThermoFisher Scientific; Jasmund et al (2007) Biomol En 24(1) 59-69), Hepatocyte Culture Medium (HCM; Lonza), Power Primary HEP Medium (Cellartis), DMEM/F12 (ThermoFisher Scientific), and William's E Medium (WEM) (ThermoFisher Scientific) (Toda et al (2020) PLos One 15 (2) e0229654; Jasmund et al (2007) Biomol En 24(1) 59-69; De Bartolo et al Biomaterials. 2006; 27: 4794-4803; Herrera et al Stem Cells. 2006; 24: 2840-2850. pmid:16945998; Ammerschlaeger et al Toxicol Sci. 2004; 78: 229-24; Yu et al Stem Cell Res. 2012; 9: 196-207; Mallanna et al Curr Protoc Stem Cell Biol. 2013; 26: 1G.4.1-1G.4.13; Cameron et al Stem Cell Reports. 2015; 5: 1250-1262; Gieseck et al PLoS One. 2014; 9: e86372; Carpentier et al Stem Cell Res. 2016; 16: 640-650); Leibovitz's L-15 medium (Sigma-Aldrich; Leibovitch (1963) Amer J. Hyg 78 173-180); Waymouth MB 752/1 medium (Sigma-Aldrich; Waymouth, Tca Manual 3, 521-525 (1977)); and SF3 (Peng et. al, IOVS 44:808-17, 2003; Jasmund et al (2007) Biomol En 24(1) 59-69) and Chee's medium (Zirvi et al Cancer Biochem Biophys. 1991 August; 12(2):137-51) In some preferred embodiments, the population of cells may be cultured in Hepatozyme.

In some preferred embodiments, the hepatocyte medium may be supplemented with β-estradiol (E2) when the set of the transcription factors comprises ERα.

The population of cells may be cultured in the hepatocyte medium under suitable conditions and for a sufficient period of time following introduction of the set of transcription factors to allow one or more cells in the population to display a hepatocyte phenotype. For example, the cells may be cultured for 10 to 40 days, preferably 20 to 30 days, for example about 20 days.

In some embodiments, the hepatocyte medium may be supplemented with one or more agents that induce expression of the set of transcription factors from encoding nucleic acid. For example, the set of transcription factors may be operably linked to one or more doxycycline inducible regulatory elements and the hepatocyte medium may be supplemented with doxycycline. The population of cells may be cultured in the hepatocyte medium supplemented with the one or more agents for 7 to 9 days, preferably 8 days. The population of cells may then be cultured in the hepatocyte medium without the one or more agents for a further 8 to 12 days, preferably 10 days.

In some embodiments, the cells in the hepatocyte medium may be further cultured in 3D. For example, the cells may be embedded in a scaffold, such as Matrigel growth factor basement membrane matrix, and cultured in hepatocyte medium, for example for 5 days or more, or 10 days or more. This may be useful for example in promoting hepatocyte functionality, such as sensitivity to cytotoxic agents.

The set of transcription factors introduced into the cell population imposes a mature hepatocyte phenotype i.e. the cells in the population are forward programmed by the set of transcription factors into hepatocytes.

The expression of one or more hepatocyte markers and/or one or more pluripotent cell markers may be monitored or detected in cells in the population during cell culture. This allows the extent of forward programming in the population to be determined as it is cultured. In some embodiments, a method may comprise identifying or confirming the identity of the hepatocytes in the culture. Hepatocytes may for example be identified in the cell culture after at least 15 days.

Following programming, the population of hepatocytes may be cultured, expanded and optionally stored, for example by cryopreservation.

The methods described above may produce a population of hepatocytes that is substantially free from other cell types. For example, a population produced by a method described herein may contain 80% or more, 85% or more, 90% or more, or 95% or more hepatocytes, following culture. The presence or proportion of hepatocytes in the population may be determined through the expression of albumin and/or α1-antitrypsin as described above. Preferably, the population of hepatocytes is sufficiently free of other cell types that no purification is required. If required, the population of hepatocytes may be purified by any convenient technique, including FACS.

Also provided is a population of hepatocytes produced from PSCs by a method described herein. At least 90%, at least 95%, at least 98%, or 100% of the population of PSCs may become hepatocytes following forward programming by a method described herein.

In some embodiments, the hepatocytes may comprise heterologous nucleic acid encoding the set of transcription factors.

Hepatocytes in the population may be functionally mature. A functionally mature hepatocyte may display a mature hepatocyte phenotype.

Hepatocytes produced by a method described herein may express the hepatocyte markers; albumin (ALB), α1-antitrypsin (AAT, A1AT or SERPINA1), CYP2A6, CYP3A4, CYP2C8, CYP2C9, UGT1A1, ApoA1, FASN, NR1H4, G6PC, UGT1A6, PCK1, PPRa/g and RORg.

Other hepatocyte markers may include fumarylacetoacetase (FAH), cytokeratin 8 (CK8), cytokeratin 18 (CK18), asialoglycoprotein Receptor (ASGR), alcohol dehydrogenase 1, arginase type I, and liver-specific organic anion transporter (LST-1).

The hepatocytes may express the hepatocyte markers at the same level or substantially the same level as primary adult human hepatocytes. For example, the expression level in the hepatocytes may be the same or higher or lower than the expression level in primary adult human hepatocytes by 20% or less, 10% or less or 5% less.

The hepatocytes may express the hepatocyte markers at levels than are higher than the expression levels in hepatocyte-like cells (HLCs) produced by directed differentiation, for example HLCs produced by a method disclosed below or in Palakkan et al., 2017; Szkolnicka & Hay, 2016; Silier et al 2015; Hay et al 2008; Baxter et al., 2015; Grandy et al., 2019; or Yiangou et al., 2018. For example, the expression level in the hepatocytes may be higher than the expression level in HLCs by 10% or more, 20% or more, 30% or more, or 50% or more.

The hepatocytes may not express progenitor markers, such as AFP, CK18 and Sox17, or may express them at low levels. For example, the level of expression of progenitor markers in the hepatocytes may be less than 20%, less than 10%, less than 5% or less than 1% of the level of expression of the above hepatocyte markers.

Preferably, the hepatocytes do not express the pluripotency associated markers, such as Oct4, Sox2, Alkaline Phosphatase, SSEA-3, Nanog, SSEA-4 and Tra-1-60, which are expressed by PSCs or display reduced expression relative to PSCs.

The expression of cell markers may be monitored and/or detected in the population of cells. For example, the expression or production of albumin (ALB), α1-antitrypsin (AAT) or other hepatocyte marker by the population of hepatocytes may be determined. This allows the extent of differentiation in the population of cultured to be determined and/or monitored. The expression of cell markers may be determined by any suitable technique, including immunocytochemistry, immunofluorescence, RT-PCR, fluorescence activated cell sorting (FACS), and enzymatic analysis.

Hepatocytes produced by a method described herein may be capable of performing the functions of primary adult human hepatocytes. For example, the hepatocytes may be able to store glycogen and LDL, synthesise and secrete AAT and/or albumin (ALB), uptake LDL and fatty acids and detoxify xenobiotics via the CytP450 pathway. The hepatocytes may be able to produce bile, thrombopoietin, angiotensinogen, urea and cholesterol; and perform glycogenolysis, gluconeogenesis, glycogenesis and lipogenesis.

A method described herein may further comprise monitoring and/or determining the ability of cells in the population to perform one or more of the above hepatocyte functions.

Hepatocyte functions may be performed by hepatocytes produced by a method described herein at same activity or substantially the same activity as primary adult human hepatocytes. For example, the amount of activity in the hepatocytes may be the same as the amount of activity in primary adult human hepatocytes or may be higher or lower by 20% or less, 10% or less or 5% less.

Hepatocytes produced by a method described herein may perform a hepatocyte function with an activity that is higher than the activity of that hepatocyte function in hepatocyte-like cells (HLCs) produced by directed differentiation, for example HLCs produced by a method disclosed Palakkan et al., 2017; Szkolnicka & Hay, 2016; Silier et al 2015; Hay et al 2008; Baxter et al., 2015; Grandy et al., 2019; or Yiangou et al., 2018. For example, the activity in the hepatocytes may be higher than the activity in HLCs by 10% or more, 20% or more, 30% or more, or 50% or more.

Hepatocytes produced by a method described herein may be capable of in vivo engraftment and the liver colonisation in model systems, for example murine mouse models, such as the humanised FRG mouse (Strom et al Methods Mol Biol 2010 640 491-509).

Hepatocytes produced by a method described herein may display the same or substantially the same gene expression profile of mature primary human hepatocytes (PHHs).

Hepatocytes produced by the present methods may display one or more of the following hepatocyte morphological characteristics: cobblestone morphology, occasional binucleity; glycogen deposits; apical microprotrusions; rough and smooth endoplasmic reticulum (ER) and a prominent Golgi body.

In some embodiments, hepatocytes which are administered to an individual may be genetically manipulated to produce a therapeutic molecule, for example a drug or growth factor (Behrstock S et al, Gene Ther 2006 March; 13(5):379-88, Klein S M et al, Hum Gene Ther 2005 April; 16(4):509-21)

A population of hepatocytes produced by the methods described herein may be used in methods of treatment of the human or animal body, for example the treatment of an individual with a liver disorder, liver injury and/or damaged or dysfunctional hepatic tissue. A population may also be used in the manufacture of a medicament for use in the treatment of a liver disorder, liver injury and/or damaged or dysfunctional hepatic tissue in an individual. A suitable individual may have an acute liver injury, for example drug induced liver injury; a chronic liver disease, such as hepatitis (e.g. hepatitis A, B, C, D, E, G or K), cirrhosis, hepatocellular carcinoma, non-alcoholic fatty liver disorder, alcoholic liver disorder, autoimmune liver disorder or an inherited metabolic disorder, such as Alpha 1 Antitrypsin deficiency, a Glycogen Storage Disease, for example Glycogen Storage Disease Type 1a, Familial Hypercholesterolemia, Hereditary Tyrosinaemia, Crigler Najjar syndrome, ornithine transcarbamylase deficiency, or factor IX deficiency or other haemophilia, haemochromatosis, Wilson's disease, Dubin-Johnson syndrome, familial amyloidosis, or Refsum's disease.

For therapeutic applications, the hepatocytes are preferably clinical grade hepatocytes.

Aspects of the invention also extend to a pharmaceutical composition, medicament, drug or other composition comprising hepatocytes produced as described herein, a method comprising administration of such hepatocytes to an individual in need thereof e.g. for treatment (which may include preventative treatment) of a liver disorder or damaged or dysfunctional hepatic tissue, as described above, and a method of making a pharmaceutical composition comprising admixing such hepatocytes with a pharmaceutically acceptable excipient, vehicle or carrier, and optionally one or more other ingredients.

A pharmaceutical composition may contain hepatocytes produced as described herein, and one or more additional components. In addition to the hepatocytes, a pharmaceutical composition may comprise a pharmaceutically acceptable excipient, carrier, buffer, preservative, stabiliser, anti-oxidant and/or other material well known to those skilled in the art. Such materials should be non-toxic and should not interfere with the activity of the hepatocytes. The precise nature of the carrier or other material will depend on the route of administration.

Liquid pharmaceutical compositions generally include a liquid carrier such as water, petroleum, animal or vegetable oils, mineral oil or synthetic oil. Physiological saline solution, tissue or cell culture media, dextrose or other saccharide solution or glycols such as ethylene glycol, propylene glycol or polyethylene glycol may be included. The composition may be in the form of a parenterally acceptable aqueous solution, which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. A composition may be prepared using artificial cerebrospinal fluid.

Hepatocytes may be implanted into a patient by any technique known in the art (e.g. Lindvall, O. (1998) Mov. Disord. 13, Suppl. 1:83-7; Freed, C. R., et al., (1997) Cell Transplant, 6, 201-202; Kordower, et al., (1995) New England Journal of Medicine, 332, 1118-1124; Freed, C. R., (1992) New England Journal of Medicine, 327, 1549-1555, Le Blanc et al, Lancet 2004 May 1; 363(9419):1439-41). In particular, cell suspensions may be injected into the portal vein of a patient. In some embodiments, the hepatocytes may be encapsulated in alginate and implanted as microbeads (Dhawan et al J. Hepatology 2020 72 5 877-884).

Administration of a pharmaceutical composition is preferably in a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors. A composition may be administered alone or in combination with other treatments, either simultaneously or sequentially dependent upon the condition to be treated.

In some embodiments, the hepatocytes in the population produced as described herein may display a normal phenotype. For example, cells may be obtained from an individual with a liver disorder or hepatic damage or dysfunction and used to produce iPS cells. In some embodiments, the iPS cells may contain a mutation or genetic defect and this mutation or defect may be corrected using conventional recombinant techniques to produce iPS cells with a normal phenotype. Hepatocytes with a normal phenotype may be produced from these iPS cells as described herein and implanted into the patient to repair or ameliorate the hepatic damage or dysfunction.

In other embodiments, the hepatocytes in the population produced as described herein may display a disease phenotype. For example, cells may be obtained from an individual with a liver disorder or hepatic damage or dysfunction and used to produce disease-specific iPS (ds-IPS) cells. Hepatocytes with a disease phenotype may be produced from these iPS cells as described herein. These cells may then be treated to restore a normal phenotype. For example, the genetic mutation or defect which is responsible for the disease phenotype may be corrected in vitro. Various techniques are available to correct genetic mutations or defects in isolated mammalian cells. Once the defect or mutation is corrected and the normal phenotype restored, the hepatocytes may be implanted into the patient to repair or ameliorate the liver disorder or hepatic damage or dysfunction.

A population of hepatocytes produced as described above may be useful in modelling the interaction of test compounds with hepatic cells, for example in toxicity screening, modelling liver disorder and screening for compounds with potential therapeutic effects.

For example, a method of screening for a compound useful in the treatment of a liver disorder may comprise;
contacting isolated hepatocyte cells produced by a method described herein with a test compound, and;
determining the effect of the test compound on said hepatocyte cells.

In some embodiments, the hepatocytes may be produced from iPSCs derived from a sample of cells with a disease associated phenotype or genotype and the effect of the test compound on the cells determined. For example, the effect on one or more disease associated pathologies may be determined.

A method of toxicology screening may comprise;
contacting isolated hepatocyte cells produced by a method described herein with a test compound, and;
determining the effect of the test compound on said hepatocyte cells or the effect of the hepatocytes on the test compound.

The effect of a test compound on growth or viability; the gene expression; or function of the hepatocytes by the determined. The growth or viability of the hepatocytes may be determined in the presence relative to the absence of the test compound. A decrease in growth or viability is indicative that the compound has a hepatotoxic effect. Gene expression may be determined in the presence relative to the absence of the test compound. For example, the expression of albumin, α1-antitrypsin (AAT), a cytochrome p450 enzyme, such as CYP3A4, CYP1A2, CYP2E1, CYP2C19, CYP2C9, and CYP2D6, factor IX, apolipoprotein A2, CEBPα and/or transthyretin, may be determined. A decrease in expression is indicative that the compound has a hepatotoxic effect. Gene expression may be determined at the nucleic acid level, for example by RT-PCR, or at the protein level, for example, by immunological techniques, such as ELISA, or by activity assays. Cytochrome p450 assays, for example, luminescent, fluorescent or chromogenic assays are well known in the art and available from commercial suppliers. One or more functions of the hepatocytes may be determined and/or measured in the presence relative to the absence of the test compound. For example, the ability of the hepatocytes to perform one or more of detoxification of organic compounds, glycogen storage, secretion of AAT or albumin, bile production, thrombopoietin production, angiotensinogen production, conversion of ammonia to urea, cholesterol synthesis, glycogenolysis, glycogenesis and lipogenesis, may be determined and/or measured. A decrease in the ability of the hepatocytes to perform one or more of these functions in the presence relative to the absence of the test compound is indicative that the compound has a hepatotoxic effect.

In some embodiments, the metabolism, degradation, or breakdown of the test compound by the hepatocytes may be determined. For example, changes in the amount or concentration of test compound and/or a metabolite of said test compound may be determined or measured over time, either continuously or at one or more time points. Decreases in the amount or concentration of test compound and/or increases in the amount or concentration of a metabolite of said test compound may be determined or measured. In some embodiments, the rate of change in the amount or concentration of test compound and/or metabolite may be determined. Suitable techniques for measuring the amount of test compound or metabolite include mass spectrometry. This may be useful in determining the in vivo half-life, toxicity, efficacy or other in vivo properties of the test compound.

Suitable hepatocytes for use in a method of screening for a compound useful in the treatment of a liver disorder or hepatic damage or dysfunction may display a disease phenotype. The effect of the test compound on one or more disease pathologies in the hepatocytes may be determined. For example, the effect of the test compound on one or more of cell growth, gene expression, protein aggregation or polymerisation; protein entrapment in the ER; cholesterol uptake; lipid and/or glycogen accumulation; and lactic acid production may be determined. Suitable techniques are well known in the art and include immunostaining, mass spectrometry, Western blots, and enzymatic assays.

A decrease or amelioration of one or more disease pathologies in the hepatocytes in the presence, relative to the absence of test compound may be indicative that the test compound may be useful in the treatment of a liver disorder or hepatic damage or dysfunction.

Methods as described herein may comprise the step of identifying a test compound which reduces or ameliorates one or more disease pathologies in the hepatocytes. Compounds which reduce disease pathologies may be useful in the development of therapeutics for the treatment of the liver disorder.

Other hepatocytes suitable for use in a method of screening for a compound useful in the treatment of a liver disorder or hepatic damage or dysfunction may display a normal phenotype and may, for example, be derived from an individual with a high risk of or high susceptibility to liver disorder, relative to the general population. The effect of the test compound on one or more of cell growth, or gene expression, for example expression of a cytochrome p450 (CYP), such as CYP3A4, CYP1A2, CYP2E1, CYP2C19, CYP2C9, and CYP2D6, may be determined. The effect of the test compound on one or more functions of the hepatocytes may be determined. For example, the ability of the hepatocytes to perform one or more of detoxification of organic compounds, glycogen storage, secretion of AAT or albumin, bile production, thrombopoietin production, angiotensinogen production, conversion of ammonia to urea, cholesterol synthesis, glycogenolysis, glycogenesis and lipogenesis, may be determined and/or measured in the presence relative to the absence of the test compound.

An increase in gene expression, growth and/or one or more functions in the presence relative to the absence of the test compound may be indicative that the compound may be useful in the treatment of a liver disorder or hepatic damage or dysfunction, such as hepatitis (e.g. hepatitis A, B, C, D, E, G or K), cirrhosis, hepatocellular carcinoma, non-alcoholic fatty liver disorder, drug induced liver injury, alcoholic liver disorder or autoimmune liver disorder.

Following identification of a compound which reduces or ameliorates one or more disease pathologies in the hepatocytes, the compound may be modified to optimise its pharmaceutical properties. This may be done using modelling techniques which are well-known in the art.

A test compound identified using one or more initial screens as having ability to reduce or ameliorate one or more disease pathologies in the hepatocytes may be assessed further using one or more secondary screens.

A secondary screen may involve testing for a biological function or activity in vitro and/or in vivo, e.g. in an animal model. For example, the ability of a test compound to reduce or ameliorate one or more symptoms or pathologies associated with the liver disorder in an animal model of the disease may be determined.

Following identification of a test compound which reduces or ameliorates one or more disease pathologies in the hepatocytes, the compound may be isolated and/or purified or alternatively it may be synthesised using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of a liver disorder as described herein.

A hepatocytes produced as described above may be useful in the disease modelling and the identification of drug targets for liver disorders.

In some embodiments, the effect of genetic mutations on the phenotype of hepatocytes produced as described above may be determined. For example, a method of identifying a genetic mutation associated with a liver disorder may comprise;
providing a test population of hepatocytes of the fifth aspect, wherein the hepatocytes in the test population each comprise a genetic mutation; and
comparing the phenotypes of the test population of hepatocytes with a control population of hepatocytes, wherein the control population does not comprise a genetic mutation; and
identifying a hepatocyte in the test population that displays a disease phenotype, such as a liver disorder phenotype,
wherein the display of a disease phenotype is indicative that the genetic mutation in the identified hepatocyte is associated with a liver disorder.

A disease phenotype is a hepatocyte phenotype that is associated with a disease, such as liver disorder. A hepatocyte with a disease phenotype may display aberrant functionality relative to a hepatocyte with a normal phenotype. For example, one or more of the hepatocyte functions described above may be reduced or absent in a hepatocyte with a disease phenotype.

In other embodiments, hepatocytes with a disease phenotype may be investigated to identify causative genetic mutations. For example, a method of identifying a genetic mutation associated with a liver disorder may comprise;
providing a test population of hepatocytes of the fifth aspect, wherein the hepatocytes in the test population display a disease phenotype, such as a liver disorder phenotype; and
comparing genomic sequence of the test population of hepatocytes with a control population of hepatocytes, wherein the hepatocytes in the control population do not display the disease phenotype; and
identifying one or more genetic mutations in the genomic sequence of the test population relative to the control population,
wherein the presence of a genetic mutation in the test population relative to the control population is indicative that the mutation is associated with the disease phenotype.

In other embodiments, hepatocytes with a disease phenotype may be investigated to identify variant gene expression. For example, a method of identifying a gene associated with a liver disorder may comprise;
providing a test population of hepatocytes of the fifth aspect, wherein the hepatocytes in the test population display a disease phenotype, such as a liver disorder phenotype; and
comparing the expression of one or more genes in the population of hepatocytes with the expression of the one or more genes in a control population of hepatocytes,
wherein a difference in the expression of a gene in the population relative to the control population is indicative that the gene is associated with a liver disorder.

A test population of hepatocytes suitable for use in these methods may be produced by a method of the first to the fourth aspects from induced pluripotent stem cells (iPSCs) derived from an individual with a liver disorder.

Also provided are methods for identifying transcription factors useful in forward programming PSCs into hepatocytes. For example, a method of identifying a transcription factor that promotes hepatocyte maturation may comprise;
determining the expression of a set of transcription factors in primary human hepatocytes (PHHs) and hepatocyte-like cells (HLCs) produced by in vitro directed differentiation, and
identifying a transcription factor in the set whose expression is increased in the PHHs relative to the CLCs,
the identified transcription factor being a candidate transcription factor for the promotion of hepatocyte maturation.

Suitable hepatocyte-like cells (HLCs) may be produced by established methods (see below or for example Palakkan et al., 2017; Szkolnicka & Hay, 2016; Silier et al 2015; Hay et al 2008; Baxter et al., 2015; Grandy et al., 2019; or Yiangou et al., 2018).

For example, the expression of one or more additional transcription factors selected from the group consisting of NR1, CUX2, AR, ZNF558, TSHZ2, TBX15, NF1X, NF1B, ATOH8, ZMAT1, ONECUT2, ZNF3858, FOS, FOSB, NR113, NPAS2, L3MBTL4, JAZF1, NF1A, ZNF680, HNF4G, CREBL2, DMRTA1, IRF6, ARID5A, SOX5, ZBTB20, ZNF704, ZEB1, ZNF367, NR1H4, KLF15, HLF and NR4A2 may be determined.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Experimental

To produce hepatocytes by forward programming, we first tested different combinations of LETFs and identify a cocktail of 3 factors sufficient to drive the conversion into immature hepatocytes (FoP-Heps). We then performed transcriptomic and epigenetic comparisons between HLCs and PHHs to identify additional transcription factors which could further increase the functional maturation of hepatocytes. This comparison revealed that a number nuclear receptors are expressed in adult hepatocytes and thus likely to be inducers of functionality and maturation in vivo. Selections of these factors were combined with LETFs and we identified that the 4TFs HNF1A-HNF6-FOXA3-RORc were the most efficient cocktail to generate FoP-Heps displaying features of mature hepatocytes including CYP3A4 activity, protein secretion and hepatotoxic response. Thus, forward programming offers an alternative to direct differentiation, bypassing the need for complex culture conditions and lengthy timelines. Moreover, FoP-Heps display a level of functionally relevant for regenerative medicine, as well as disease modelling or drug screening.

### Materials and Methods

hPSC Culture

The human ESC H9 (WiCell) and IPSC A1ATDR/R, FS13B and NIH Lrg1 (Yusa et al., 2011) lines were used in this project. Human iPSC line was derived as previously described, under approval by the regional research ethics committee (REC 08/H0311/201). Both hPSCs were cultured on vitronectin XFTM (10 μg/mL, StemCell Technologies)-coated plates and in Essential 8 (E8) chemically defined medium consisting of DMEM/F12 (Gibco), L-ascorbic acid 2-phosphate (1%), insulin-transferrin-selenium solution (2%, Life Technologies), sodium bicarbonate (0.7%), and Penicillin/Streptomycin (1%), freshly supplemented with TGFB (10 ng/ml, R&D) and FGF2 (12 ng/ml, Qkine) (Chen et al., 2011). For routine dissociation, cells were incubated with 0.5 μM EDTA (ThermoFisher Scientific) for 3 minutes at 37° C. seeded in small clumps. Cells were maintained at 37° C. in 20% 02, 5% CO2 and medium was replenished every 24 hours.

### Gene Targeting

Inducible hESC and hiPSC lines were generated using the OPTi-OX system as previously described (Bertero et al., 2016; Pawlowski et al., 2017). Briefly, two gene safe harbours were targeted (GSH). The hROSA26 locus was targeted with a constitutively expressed transactivator (rtTA) and the AAVS1 locus with the transgenes of interest under a TET responsive element (TRE). Different combinations of transcription factors and/or nuclear receptors as stated throughout the manuscript were cloned. Template cDNA sequences were obtained either from Dharmacon: HNF6 (MHS6278-213244170), HNF1A (MHS6278-202857902), RORy (MHS6278-202800991) and ESR1 (MHS6278-211691051); or amplified from human primary liver cDNA: HNF4A, FOXA3 and AR. Sequences were amplified using the KAPA HiFi HotStart ReadyMix (Roche). The primers used to amplify and clone the sequences into the backbone vector contained upstream and downstream overhangs in order to generate a GSG (Gly-Ser-Gly) linker and a different 2A peptide as listed in table 1. The different vectors were constructed by Gibson Assembly (New England Biolabs) using a 1:3 pmol ratio of vector to insert. For targeting, hPSCs were dissociated into single cells with STEMpro accutase (Thermo Fisher) for 5 minutes, and 1 million cells were transfected with 2 μg of donor vector and 2 μg of each AAVS1 ZFN expression plasmids using the P3 Primary Cell 4D-Nucleofector X Kit (Lonza). Cells were seeded in E8 medium supplemented with 10 μM ROCK Inhibitor Y-27632 (Selleckchem). After 5-7 days, colonies were selected with 1 μg/ml puromycin (Sigma Aldrich) for at least 2 days, after which they were individually picked and genotyped as previously described (Bertero et al., 2016; Pawlowski et al., 2017).

### Hepatocyte Direct Differentiation

hPSCs were dissociated into single cells following incubation with StemPro Accutase (Thermo Fisher) for 5 minutes at 37° C. and seeded at a density of 50.000 cells/cm2 in E8 medium supplemented with 10 μM ROCK Inhibitor Y-27632 (Selleckchem). Hepatocytes were differentiated 48 h after seeding, as previously reported (Hannan et al., 2013) with minor modifications. Following endoderm differentiation, anterior foregut specification was achieved with RPMI-B27 differentiation media supplemented with 50 ng/ml Activin A (R&D) for 5 days. Cells at the foregut stage were further differentiated into hepatocytes with Hepatozyme complete medium: HepatoZYME-SFM (Thermo Fisher) supplemented with 2 mM L-glutamine (Thermo Fisher), 1% penicillin-streptomycin (Thermo Fisher), 2% non-essential amino acids (Thermo Fisher), 2% chemically defined lipids (Thermo Fisher), 14 μg/ml of insulin (Roche), 30 μg/ml of transferrin (Roche), 50 ng/ml hepatocyte growth factor (R&D), and 20 ng/ml oncostatin M (R&D), for up to 27 days.

Forward Programming into Hepatocytes

hPSCs were dissociated into single cells following incubation with StemPro Accutase (Thermo Fisher) for 5 minutes at 37° C. and seeded at a density of 40-50.000 cells/cm2 in E8 medium supplemented with 10 μM ROCK Inhibitor Y-27632 (Selleckchem). E8 medium was replenished the following day. Following 48 h, initial induction of the transgenes was achieved by incubation in E6 medium (E8 without growth factors) supplemented with 1 mg/ml doxycycline (dox) for 24 h. Cells were then maintained in Hepatozyme complete medium supplemented with 1 mg/ml dox for the remaining duration of the protocol. Medium was replenished every day for the next 4 days, and every other day here after. For specific experiments, cell lines were treated with 100 nM of desmosterol, testosterone or p3-estradiol (E2) from day 2 of forward programming. All ligands were purchased from Sigma-Aldrich and reconstituted in ethanol. For 3D cultures, forward programmed cells were embedded in Matrigel Growth Factor Reduced Basement Membrane Matrix, Phenol Red-free (Corning) at day 15 or day 20 and cultured for 5 days or 10 days, respectively. Cells were dissociated with Hank's based cell dissociation buffer (Gibco) for 20 minutes at 37° C., resuspended in Matrigel and seeded in 40-50 μL domes in Hepatozyme complete medium supplemented with 1 mg/ml dox.

### Primary Human Hepatocytes

Fresh primary hepatocytes used for RNA-seq were obtained as previously reported (Segeritz et al., 2018). Primary plated hepatocytes from 4 donors (3 males and 1 female) were purchased from Biopredic International (Rennes, France), meting the manufacturer's quality control requirements. Cells were maintained in short-term monolayer cultures in William's E (Gibco) supplemented with 1% Glutamine (Gibco), 1% Penicillin-streptomycin (Gibco), 700 nM Insulin (Sigma-Aldrich) and 50 μM Hydrocortisone (Sigma). Functional assays such as CYP3A4 activity measurement were performed in Hepatozyme complete medium within 8 h of receipt.

### CYP3A4 Assay

Measurement of CYP3A4 enzymatic activity was performed using the P450 Glo kit (Promega). Cells were incubate with 1:1000 luciferin-IPA in Hepatozyme complete for 1 h at 37° C. Supernatant was mixed with detection reagent in a 1:1 ratio and incubated at RT for 20 minutes in Greiner white 96 well microplates (Sigma Aldrich). Luminescence was measured in triplicate on a GloMax plate reader. Hepatozyme complete medium was used as background control. Relative light units were normalised for background, volume and average total number of cells obtained after differentiation.

### LDL Uptake Assay

LDL uptake capacity was measured with the LDL Uptake Assay Kit (Abcam). Cells were incubated with 1:100 human LDL conjugated to DyLight™ 550 in Hepatozyme complete medium for 3 hours at 37° C. Cells were then washed and fixed with 4% PFA for 20 minutes at 4° C.

### Fatty Acid Treatments

Forward programmed cells were embedded in 3D from day 20 and cultured for 7 days in Hepatozyme complete medium supplemented with either BSA (control), or oleic acid (0.25 mM) or palmitic acid (0.25 mM) conjugated with BSA. Intracellular lipid accumulation was detected by incubating cells with 1 μl/ml Bodipy (Thermo scientific) for 30 minutes, followed by DAPI (Hoechst) diluted 1:10.000 in PBS for 30 minutes and imaged on a Zeiss LSM 700 confocal microscope.

### APAP Toxicity

The hepatotoxicity of acetaminophen (APAP) was tested by incubating forward programmed cells cultured in 3D from day 15 in Hepatozyme complete medium supplemented with 25 mM acetaminophen (R&D) for 48 h hours (day 18 to day 20) after which cell viability was determined.

### Cell Viability

Cell viability was determined by incubating cells with 1:10 Presto Blue reagent (Invitrogen) in Hepatozyme complete medium at 37° C. for 4 hours. Fluorescence was measured using the EnVision plate reader with an excitation emission of 560 nm/590 nm.

### RT-qPCR

RNA was extracted from either cells or tissues using GenElute Mammalian Total RNA Miniprep Kit (Sigma-Aldrich) according to the manufacturer's instruction. 500 ng of RNA were reverse transcribed into cDNA using Random Primers and SuperScript II (Invitrogen) according to the manufacturer's instructions. qPCR was performed using the KAPA SYBR FAST qPCR Kit low-ROX (Sigma-Aldrich) with 200 nM of forward and reverse primers (Sigma-Aldrich; primers listed in table 2) on a QuantStudio 5 (Applied Biosystems). qPCRs were performed in technical duplicates and normalised to the average of two housekeeping genes (RPLP0 and PBGD) using the 2-ΔCt method.

### Immunofluorescence Staining

Cells in monolayer were fixed in 4% PFA for 20 minutes at 4° C. and blocked for 30 minutes in 10% donkey serum (BioRad) and 0.1% Triton X-100 (Sigma-Aldrich). Fixed cells were incubated with primary antibodies listed in table 3 in 1% donkey serum and 0.01% Triton X-100 overnight at 4° C. Following washing, cells were incubated with Alexa Fluor 488-, 568- or 647-conjugated secondary antibodies (Life Technologies) for 1 h at room temperature diluted in 1% donkey serum and 0.01% Triton X-100. For nuclei visualisation, cells were incubated with adding DAPI/Hoechst 33258 (bis-Benzimide H, Sigma-Aldrich) diluted 1:10.000 in PBS for 10 minutes at room temperature. Cells were imaged either on a Zeiss Axiovert 200M or on a Zeiss LSM 700 confocal microscope.

### Secreted Protein Quantification

Albumin, alpha-fetoprotein and alpha-1-antitrypsin were measured in the cell culture supernatant of monolayer cultures, which were replenished with fresh Hepatozyme complete medium 24 h prior to collection. Concentrations were detected on by ELISA (performed by core biomedical assay laboratory, Cambridge University Hospitals) and normalised to cell number.

### RNA-Seq Analyses

RNA-seq datasets were generated for undifferentiated hiPSCs (n=3), hESC-derived HLCs (n=2), hiPSC-derived HLCs (n=6), freshly harvested PHHs (fPHHs, n=3) and commercially purchased PHHs (pPHHs, n=2). RNA was extracted from either cells or tissues using GenElute Mammalian Total RNA Miniprep Kit (Sigma-Aldrich) according to the manufacturer's instruction. Poly-A library preparation and sequencing were performed by Cambridge Genomic Services (hESC_HLCs; pPHHs) and the Wellcome Trust Sanger Institute (hiPSCs, hiPSC_HLCs, fPHHs). Quality of reads was assessed with FastQC. For consistency, fastq reads were split into single-end reads and trimmed to the same length (40 bp) using cutadapt version 2.10. Single-end fastq files were mapped and quantified using salmon version 1.2.1 with the following parameters: -l A, -GCbias, -posbias, -validatemappings (Patro et al., 2017). The index used was pre-built from the human GRCh38 cDNA reference sequence from Ensembl (refgenomes.databio.org). Differential gene expression was calculated using DESeq2 (Love et al., 2014), with the following parameters padj>0.05, basemean >100 and log 2 fold change >2 or <−2 between groups as depicted in each figure. Gene ontology enrichment was calculated with the clusterProfiler package (Yu et al., 2012). Pathway analysis on significantly misregulated transcription factors was assessed using ReactomePA (Yu & He, 2016). Mouse liver polyA plus RNA-seq was downloaded from ENCODE (Consortium, 2012). Single-end fastq reads were trimmed in both replicates from each dataset to 70 bp using cutadapt version 2.10. Fastq were mapped and quantified using salmon version 1.2.1 with the following parameters: -I A, -Gbias, -seqbias, -validatemappings using a pre-build mm10 cDNA reference genome. DeSeq2 was used to generate all plots for visualisation.

### Chromatin Immunoprecipitation (ChIP)

ChIP was preformed has previously reported (Brown et al., 2011). Briefly, chromatin was crosslinked with 1% formaldehyde (Sigma-Aldrich) for 10 minutes at room temperature and quenched with 0.125M glycine (Sigma-Aldrich). Cells and nuclei were subsequently lysed and chromatin was sonicated to fragment DNA to about 200-500 bp on a Bioruptor Pico sonication device (Diagenode). Sonicated chromatin was pre-cleared with same-host IgG and protein G Dynabeads (Thermo Fisher), 100 μg of cleared chromatin (protein) was incubated with 2 μg of the following antibodies overnight at 4° C.: H3K27ac (Abcam, ab4729), H3K4me1 (Abcam, ab8895), H3K27me3 (Active Motif, 39155) and H3K4me3 (Merk, 05-745R), after which complexes captured with 30 μl of protein G Dynabeads (Thermo Fisher). Complexes were washed, RNAse A (Thermo Fisher) and Proteinase K (Sigma-Aldrich) treated and DNA was purified by phenol-chloroform extraction and precipitated with GlycoBlue (Thermo Fisher), sodium acetate (Thermo Fisher) and ethanol (Sigma-Aldrich). A sonicated chromatin sample (1%) was also collected as input for normalisation and 10 ng of DNA were used for ChIP-sequencing library preparations.

### ChIP-Seq Analyses

Library preparation and sequencing and alignment were performed by the Wellcome Trust Sanger Institute DNA Sequencing Facility (Hinxton, UK). Sequencing was performed on an Illumina HiSeq v4 to obtain paired-end reads with 75 bp length. ChIP-seq reads were mapped to human genome assembly GRCh38 with bwa. Aligned data in BAM format was sorted and indexed with samtools. Coverage files were generated using deeptools bamCoverage, with a binsize of 10 bp and normalised as RPKM for visualisation in IGV and heatmap representation with deeptools. In order to plot principal component analysis (PCA), average scores were calculated over 1000 bp bins. For peak calling, BAM files were converted to SAM and peaks called using homer (Heinz et al., 2010). Both replicates were used for peak calling against input with disabled local filtering invoking the following flags for H3K27ac: -region -L 0. In order to identify regulatory regions specifically active in PHH or HLCs, differentially bound peaks were determined using PHH datasets as target against all HLCs datasets as background, and vice versa, with a fold enrichment over background of 4. For motif enrichment, peak calling was performed on nucleosome free regions by invoking the flags -L 1 -nfr, in order to determine the “dips” within H3K27ac-rich regions. These sets of regions were overlapped with the differentially bound peaks as above, in order to preform PHH or HLC-specific motif enrichment. Peak annotation and gene ontology enrichment were determined with the clusterProfiler R package (Yu et al., 2012). Undifferentiated hiPSCs ChIP-seq reads aligned to the same genome assembly were downloaded from ENCODE (Consortium, 2012) and treated as above.

### Data Availability

RNA-seq datasets used in this study are accessible on Array Express under the accession number E-MTAB-10634. In addition, 3 of the hiPSC_HLCs data sets have been previously deposited with the accession number E-MTAB-6781 (Segeritz et al., 2018). Mus musculus C57BL/6 liver embryo RNA-seq datasets were obtained from the ENCODE database (Nakamori et al., 2016) (encodeproject.org) with the following accession numbers: ENCSR216KLZ (E12.5 liver), ENCSR826HIQ (E16.5 liver), ENCSR096STK (P0 liver), ENCSR000BYS (8 weeks mixed sex adult liver) and ENCSR216KLZ (10 weeks adult liver). ChIP-seq datasets generated in this study have been deposited on Array Express with the accession number E-MTAB-10637, and publicly available datasets for hiPSCs were used from the ENCODE database with the following accession numbers: ENCSR729ENO (H3K27ac), ENCSR249YGG (H3K4me1), ENCSR386RIJ (H3K27me3), ENCSR657DYL (H3K4me3) and ENCSR773IYZ (input).

### Statistical Analysis

Statistical analyses were conducted using GraphPad 9.0.0 and specific tests are indicated in the figure legends. For each figure, sample size n indicates to the number of independent experiments or biological replicates and individual values are represented for every graph. Testing between groups was performed with at least n≥3 independent experiments and exact p values are indicated within the figure where significant.

### Results

Liver-Enriched Transcriptions Factors Allow Forward Programming into Cells with Hepatocyte Identity

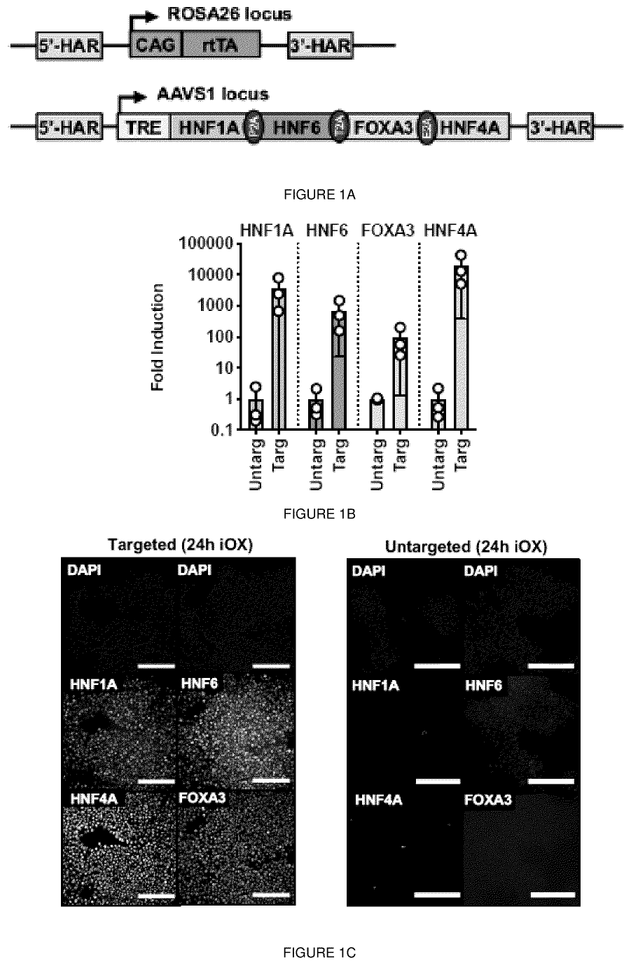

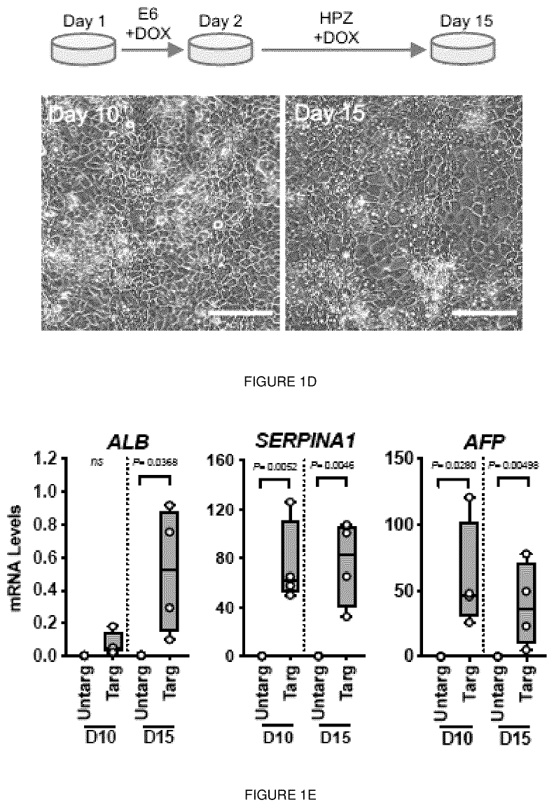

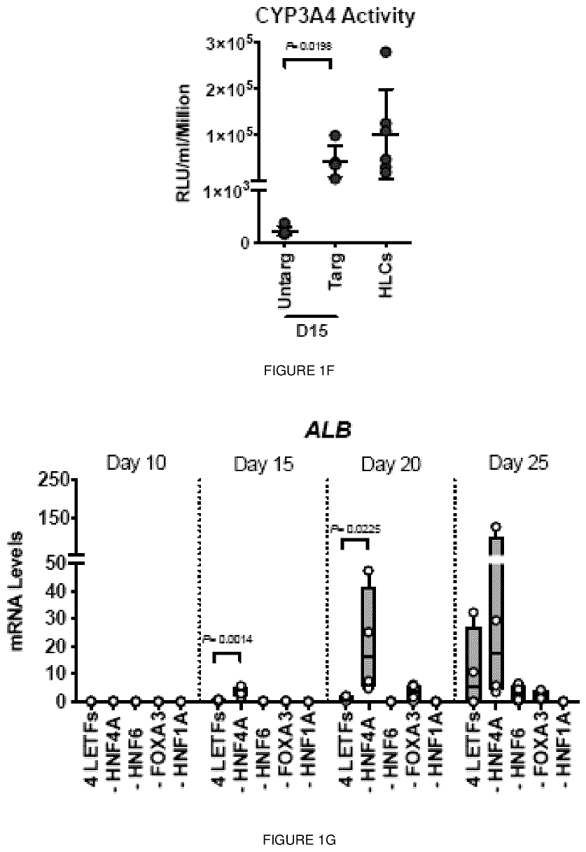

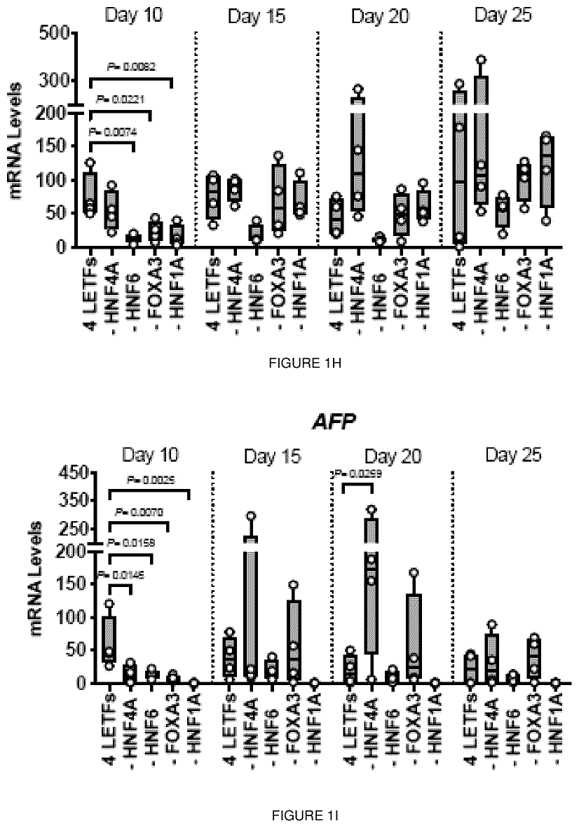

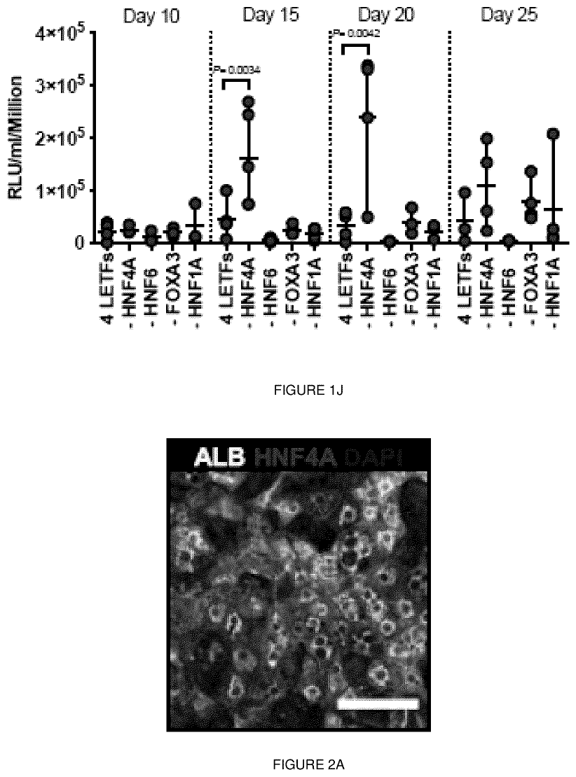

The first step to develop a forward programming method consists in identifying a cocktail of transcription factors which can recreate the transcriptional network characterising the target cell type. However, this step is challenging for hepatocytes as liver development is not initiated by a single and specific master regulator, and the factors driving functional maturation of hepatocytes remain to be fully uncovered. To bypass these limitations, we decided to focus on the LETFs which are known to control the induction of the hepatic program during foetal development and have been tested in somatic cell conversion (Rombaut et al., 2021). The coding sequence of 4 LETFs (HNF4A, HNF1A, HNF6 and FOXA3) was cloned into the OPTi-OX system (FIG. 1A) and the resulting inducible cassette was targeted into the AVSS1 gene safe harbour (Bertero et al., 2016; Pawlowski et al., 2017). After selection, individual sublines were picked, expanded and genotyped before further characterisation. Addition of doxycycline (dox) for 24 hrs was sufficient to induce homogenous and robust expression of each LETF in the selected hESCs (FIGS. 1B and 1C)

confirming the efficacy of the OPTi-OX system in inducing transgene expression. Importantly, this induction was not associated with differentiation into liver cells, suggesting that LETFs alone are not sufficient to impose an hepatocytic identity. Thus, we decided to screen culture conditions which could sustained both the survival and differentiation of hepatocytes (data not shown) and found that after the initial 24 h in E6 medium, the cells acquired an hepatocyte-like morphology when cultured in Hepatozyme complete medium for 14 days (FIG. 1D). Interestingly, the resulting cells expressed hepatocyte markers such as Albumin (ALB), Alpha-1 Antitrypsin (A1AT or SERPINA1) and Alpha-Fetoprotein (AFP) (FIG. 1E) and display CYP3A4 activity levels comparable to HLCs generated by direct differentiation (FIG. 1F). Next, we asked whether all the 4 LETFs were necessary to achieve this hepatocyte-like phenotype. For that, we removed each factor to generate hESCs sublines expressing combinations of 3 factors. Robust and homogeneous expression at the protein level was again confirmed after 24 h of dox induction. Induction of each combination of 3 LETFs in culture conditions identified above showed that HNF1A, HNF6 or FOXA3 were necessary to generate cells expressing hepatocytes markers such ALB (FIG. 1G). HNF4A overexpression seemed to be dispensable as cells generated by overexpression of the 3 remaining LETFs (HNF1A, HNF6, FOXA3) acquired a cobblestone-like morphology, expressed high levels of ALB, SERPINA1 and AFP (FIG. 1G, 1H, and 1I). Strikingly, hepatocytes generated using these 3TFs (3TF FoP-Heps) achieved the highest levels of CYP3A4 activity suggesting overexpression of HNF4A could block the acquisition of functional characteristics (FIG. 1J). Altogether, these results showed that overexpression of HNF1A, HNF6 and FOXA3, is sufficient to forward program hPSCs into hepatocyte-like cells.

### HLCs Generated by Direct Differentiation Lack the Expression of Specific Nuclear Receptors

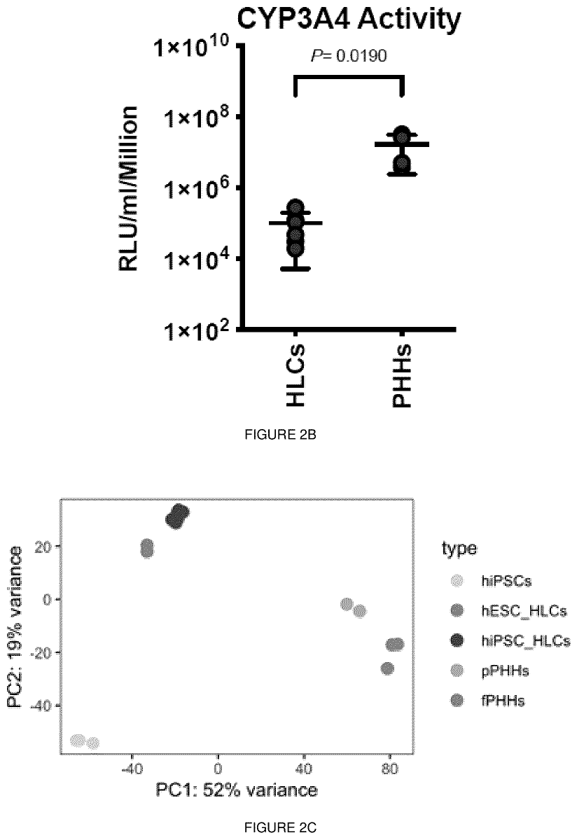

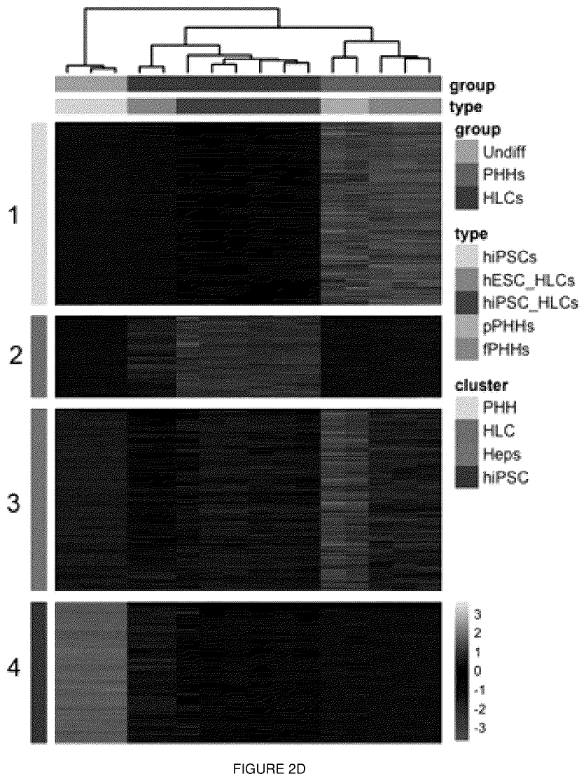

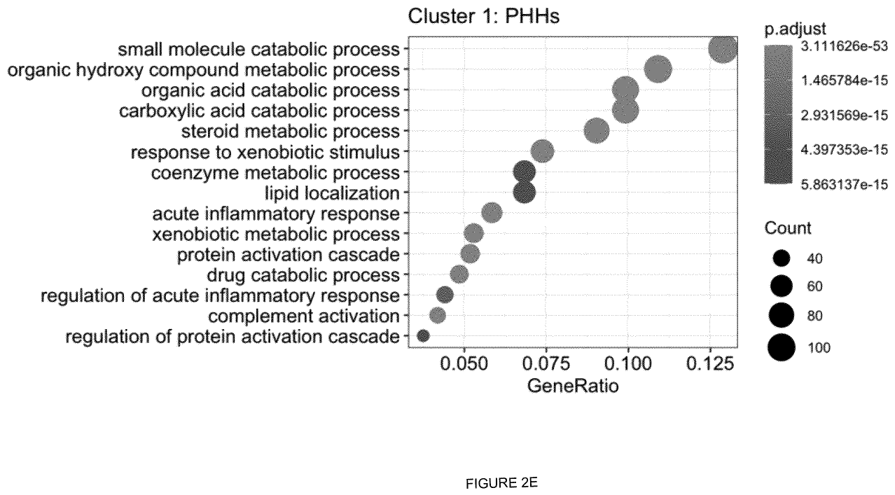

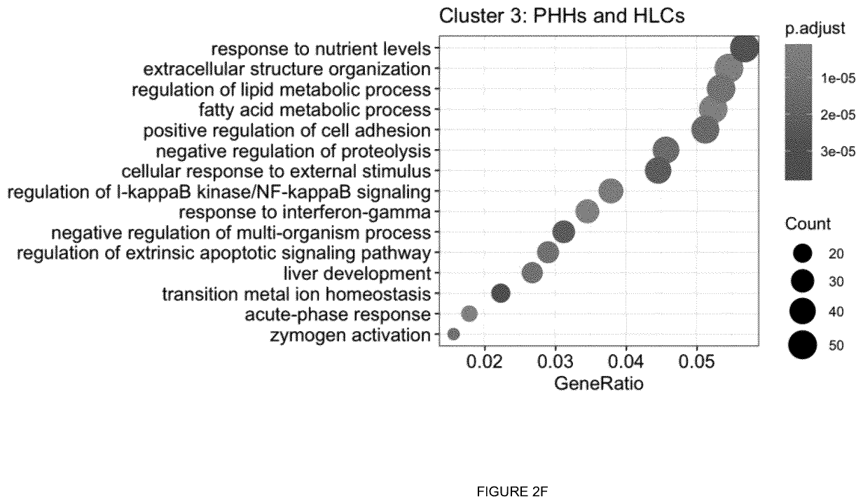

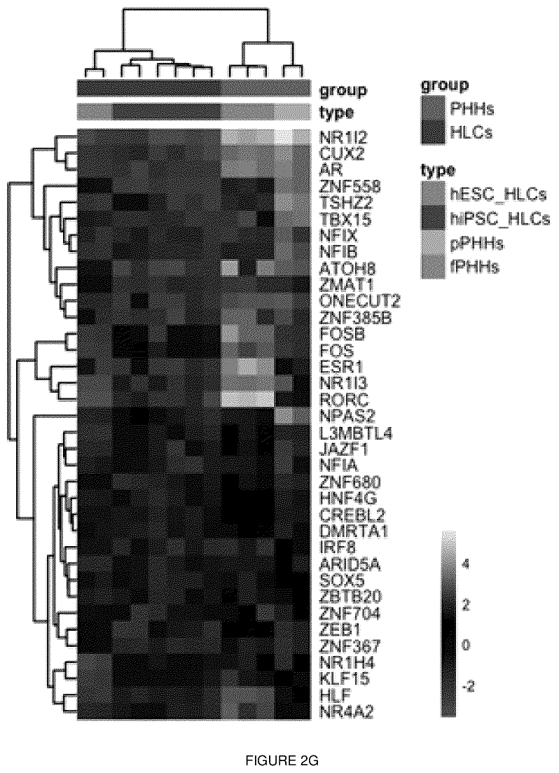

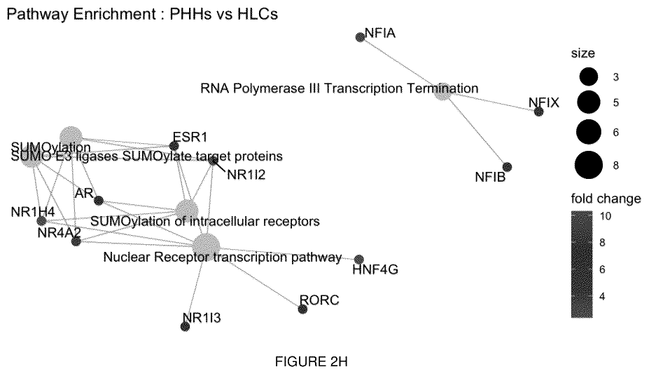

Following these encouraging results, we aimed to increase the functionality of 3TF FoP-Heps by adding TFs which could play a role in promoting hepatic maturation and functionality. However, identifying these factors proved to be challenging as there is little information about the mechanisms driving functional maturation of hepatocytes, especially around the neonatal stage when adult hepatic functions are established. To bypass this limitation, we decided to compare the transcriptome profile of adult PHHs to the transcriptome of HLCs generated from hPSCs by direct differentiation. Indeed, HLCs represent a foetal state which has been broadly characterised (Baxter et al., 2015), while 3TFs FoP-Heps are likely to be less relevant for natural development. For this comparison, we used a state-of-the-art protocol (Hannan et al., 2013; Touboul et al., 2010) which has been used for modelling liver disorder (Rashid et al., 2010; Segeritz et al., 2018) and as proof of concept for cell-based therapy applications (Yusa et al., 2011). This protocol starts by the production of endoderm cells expressing SOX17, followed by the specification of foregut expressing HHEX, after which cells transit through an hepatoblast-like state marked by TBX3. Interestingly, LETFs were expressed during this differentiation at levels comparable to PHHs confirming that these first steps follow a natural path of development. The resulting progenitors undergo a final stage of differentiation into HLCs expressing functional markers such as ALB and SERPINA1 (FIG. 2A). Despite displaying key hepatic functions (Baxter et al., 2015; Grandy et al., 2019; Yiangou et al., 2018) HLCs represent a “foetal” state as shown by the expression of AFP or by the limited activity/expression of CYP3A4, CYP2A6 or CYP2C9 (FIG. 1B). RNA-sequencing (RNA-seq) performed on HLCs generated from either human induced pluripotent stem cells (hiPSCs) or human embryonic stem cells (hESCs), and from PHHs) freshly harvested (fPHHs) or cultured in vitro as monolayer (pPHHs) reinforce these observations. Principal component analysis (PCA) of the most variable 500 genes showed a clear distinction between the 3 cell types, with HLCs clustering in between undifferentiated hiPSCs and PHHs confirming their intermediate state of differentiation (PC1: 52%, FIG. 2C). In order to further explore the differences between HLCs and PHHs, we combined Differential Gene Expression (DGE) and Gene Ontology (GO) analyses to identify genes and biological functions specific to each cell type (FIG. 2D). Genes uniquely expressed in PHHs (cluster 1) were associated with adult liver functions such as response to xenobiotic and xenobiotic metabolism, inflammatory response and complement activation (FIG. 2E). Genes expressed in both HLCs and PHHs (Heps; cluster 3) were involved in liver development, fatty acid metabolism or broad cellular functions (FIG. 1F). Of note, genes specifically up-regulated in HLCs were associated with extracellular matrix organization and varied developmental functions which could originate from their in vitro environment. We then decided to focus specifically on transcription factors (TFs) using a previously curated list (Lambert et al., 2018) (FIGS. 2G and 2H) and identified 36 TFs highly expressed in PHHs vs HLCs (p<0.05, log 2 fold change >2). Interestingly, reactome pathway analysis grouped these TFs into two main pathways: the NFI family and a cohort of 8 nuclear receptors (FIG. 2H) which are known to play a role in liver metabolic activity. Taken together these observations show that “foetal” state of HLCs is associated with the absence of several nuclear receptors thereby suggesting that these factors could be necessary to drive functional maturation of hepatocytes.

### Epigenetic Characterisation of HLCs Suggests a Role for Nuclear Receptors RORc, AR and ERα

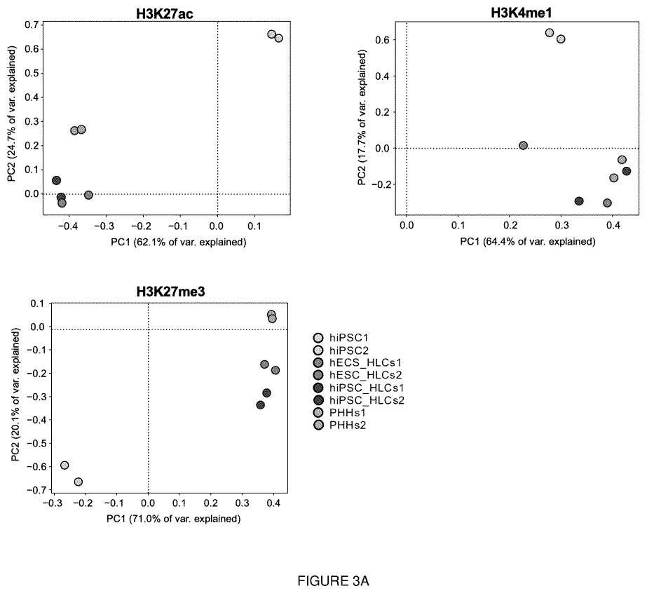

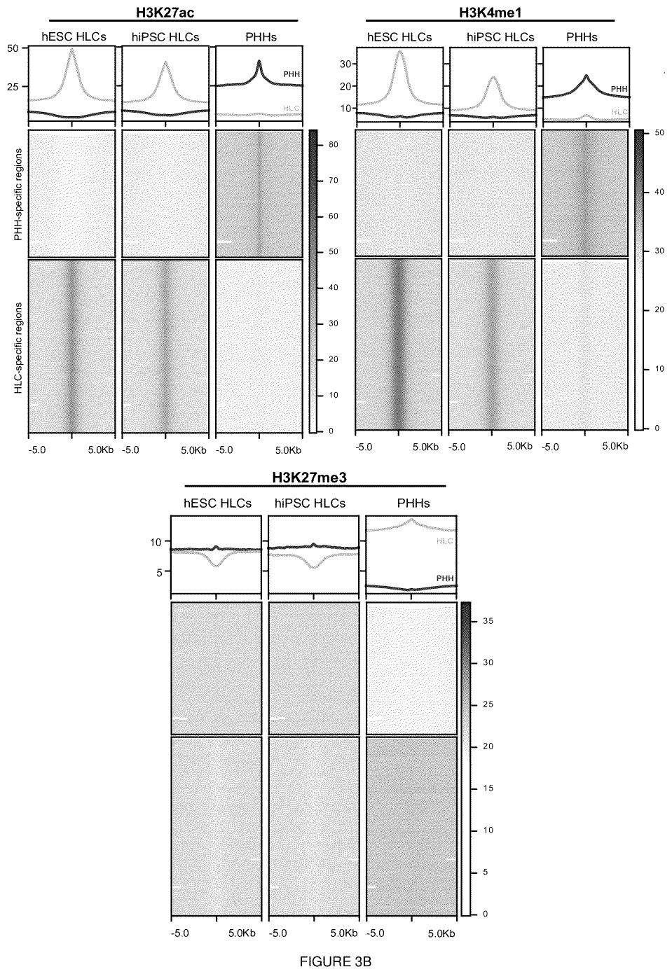

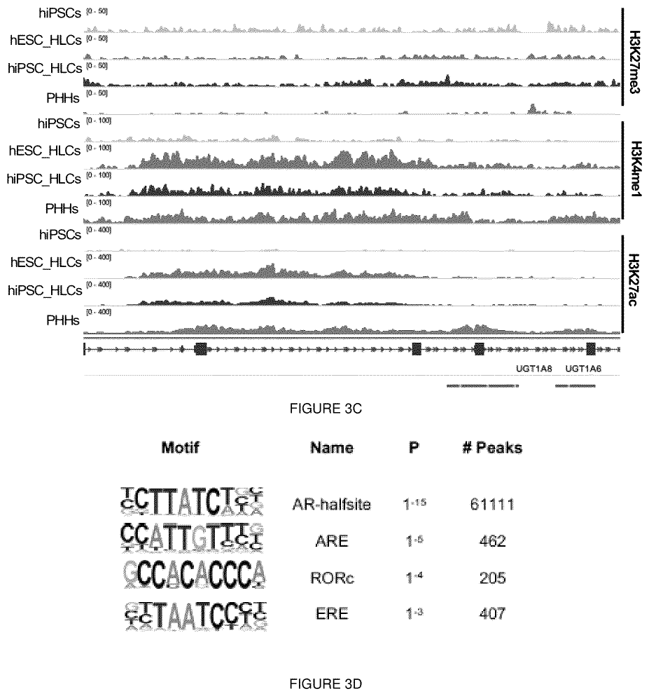

To further refine the list of TFs identified by our transcriptomic analyses, we decided to compare the epigenetic landscape of HLCs vs PHHs. Indeed, we hypothesised that TFs binding regulatory regions in PHHs could have a key function in maturation. ChIP-sequencing (ChIP-seq) was performed on histone marks including H3K27ac (active regulatory regions), H3K4me1 (active or primed regulatory regions), and H3K27me3 (silenced genes) (Creyghton et al., 2010; Wang et al., 2015). These marks were profiled in HLCs derived from both hiPSCs and hESCs, and PHHs while undifferentiated hiPSCs were used as control. As expected, PCA analyses showed a marked divergence between the epigenetic profile of HLCs and hiPSCs independently of the mark analysed (FIG. 3A). Interestingly, HLCs and PHHs clustered in close proximity suggesting that these cell types share an important part of their epigenetic profile despite their transcriptomic differences. Analyses of H3K27ac provided the strongest distinction between HLCs and their natural counterparts, confirming the importance of this mark for establishing cellular identity (FIG. 3A) and suggesting that H3K27ac could be the most informative mark in understanding the divergence between HLCs and PHHs. We then performed differential peak calling to identify regulatory regions uniquely enriched and active in PHHs versus HLCs (“PHH-specific”), and vice versa (“HLC-specific”). We profiled H3K4me1 and H3K27me3 in either PHH or HLC-specific regions. This analysis revealed that H3K4me1 was absent at “PHH-specific” regions in HLCs and seems to be broadly replaced by spread of H3K27me3 deposition instead (FIG. 3B). Interestingly, discrete portions of regulatory regions lacked H3K27ac in gene down regulated in HLCs (see for example CYP3A4 and UGT1A, FIG. 3C). Ontology of genes associated with each set of regions highlighted several adult liver metabolic processes in the “PHH-specific” set such as steroid, lipid and xenobiotic metabolism, and range of developmental functions for “HLC-unique” regions, in agreement with the transcriptomic analyses. Overall, these results suggested that a subset of genes involved in adult liver functions lack H3K4me1 priming as well as full H3K27ac deposition in HLCs.

These gene can also display repressive marks such as H3K27me3. In addition, HLCs display active histone marks in regions including genes which are not associated with liver differentiation confirming that cells generated from hPSCs also present an epigenetic signature specific to their in vitro state. Taken together these observations suggested that HLCs and PHHs broadly share the same epigenetic identity. However, the activation of a limited and specific set of regulatory regions is missing in HLCs, which explains their lack of functional maturation. To identify the nuclear receptors potentially involved in regulating these regions, we performed motif enrichment analysis in the “PHH-specific” regions marked by H3K27ac. Interestingly, this analysis identified a significant enrichment for the androgen (AR) and Estrogen (ERα) response elements, as well as RORc motifs (FIG. 3D), which were among the top differentially expressed nuclear receptors in our transcriptomic analyses. We then decided to further investigate the importance of these nuclear receptors throughout development using mouse RNA-seq datasets obtained at different stages of liver organogenesis (E12.5, E16.5, P0, 8 week and 10 week adults. Interestingly, the expression of these 3 nuclear receptors was found to be upregulated specifically in the adult liver. Altogether, these observations suggested that the nuclear receptors AR, ERα and RORc could play a role in establishing or maintaining a transcriptional network characterising mature hepatocytes.

### Overexpression of RORc Increases the Functionality of Hepatocytes Generated by Forward Programming

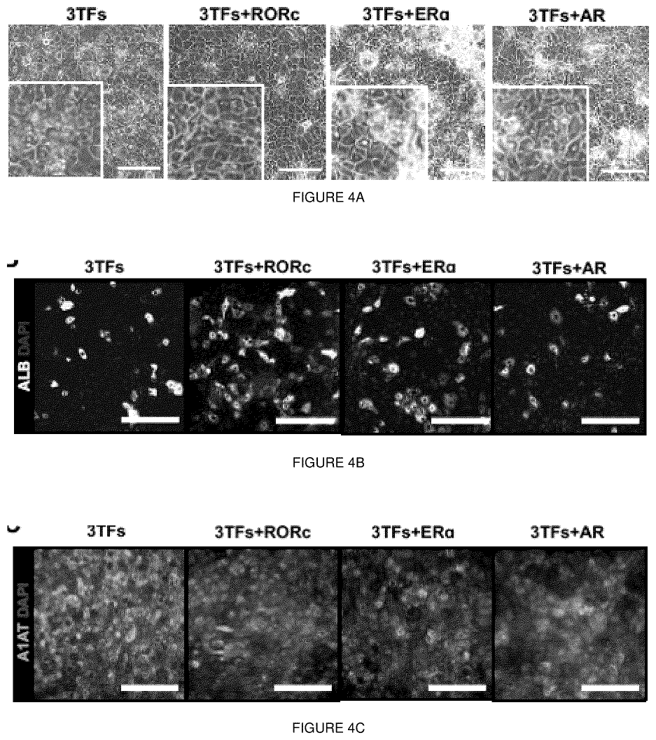

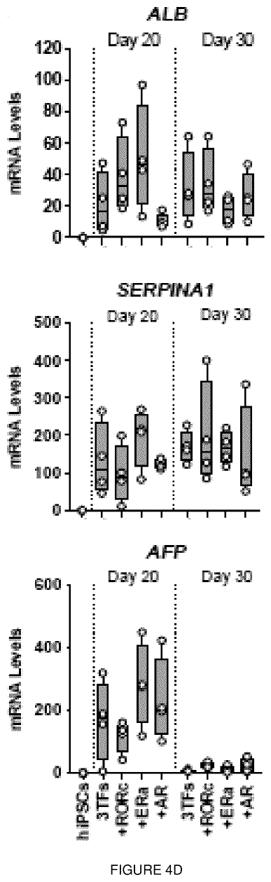

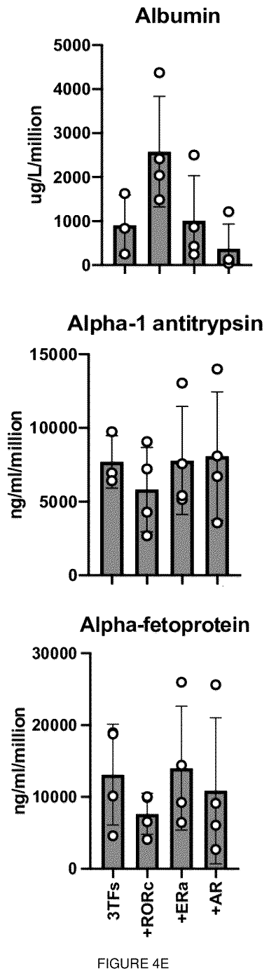

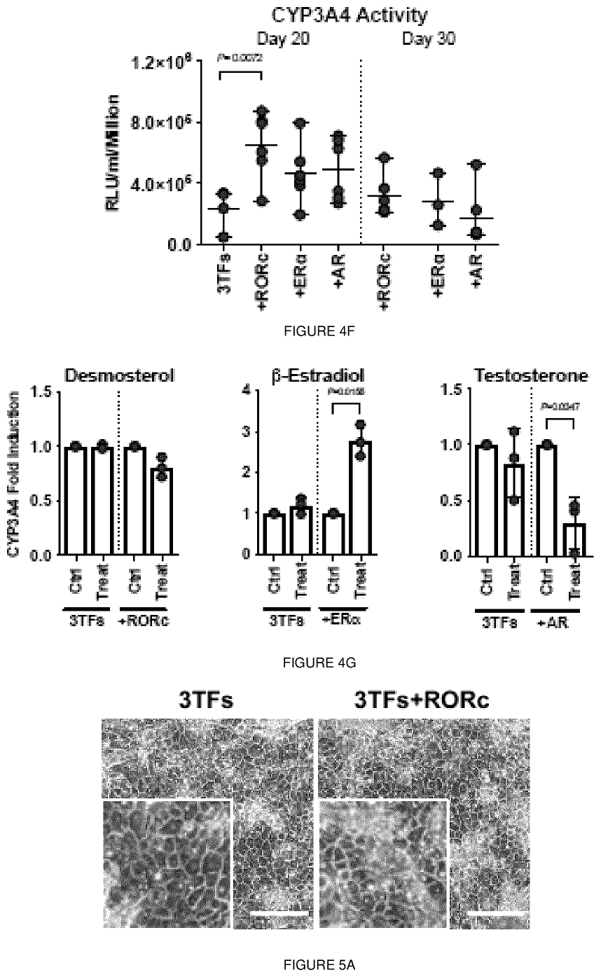

We next tested the capacity of RORc (RORy), AR, and ERα (ESR1), to further improve the functionality of FoP-Heps generated using 3 LETFS. For that, we generated hESC lines inducible for the expression HNF1A, HNF6 and FOXA3 (3TFs) in combination with each of the nuclear receptors identified above. The homogeneous induction of the 4TFs was validated using immunostaining. Interestingly, these analyses showed that the overexpressed nuclear receptors were located in nucleus suggesting that their overexpression might bypass the need of ligands to promote their activity. We then induced forward programming using the culture conditions identified above and observed the production of polyploid cells with a cobblestone morphology (FIG. 4A). The hepatocytic identity of these cells was confirmed by the expression of Albumin, SERPINA1/A1AT and AFP in all lines (FIGS. 4B, 4C, 4D, and 4E). Notably, RORc overexpression resulted in cells with the highest Albumin protein levels (FIGS. 4B, 4D, and 4E) and lower level of AFP expression especially after 30 days of differentiation (FIGS. 4D and 4E). CYP3A4 activity levels were also significantly higher in cells generated in the presence of RORc, as compared with cells reprogrammed with only 3TFs (FIG. 4F). We next tested whether stimulation with exogenous ligands specific for each nuclear receptor could further induce functional maturation as measured by CYP3A4 activity (desmosterol for RORc, p3-estradiol for ERα and testosterone for AR). Interestingly, only p3-estradiol treatment resulted in a 3-fold increase in CYP3A4 activity, whereas testosterone treatment significantly decreased CYP3A4 activity and desmosterol had no effect (FIG. 4G). This increase was not observed with 3TFs Fop-Heps thereby suggesting the effect of these ligands was linked to the overexpression of their receptor.

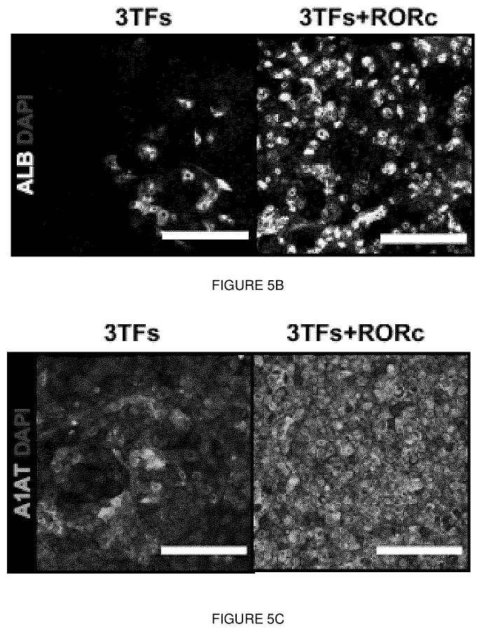

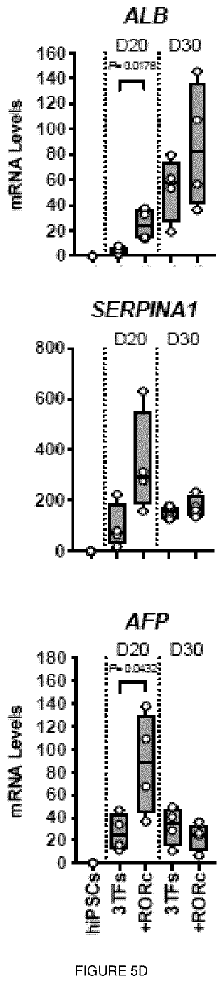

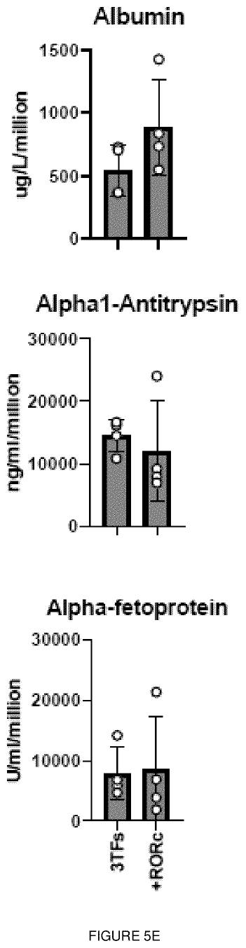

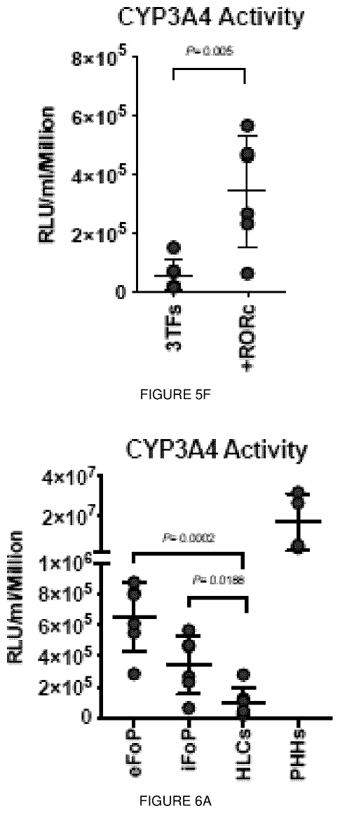

RORc generated FoP-Heps appeared to have the highest level of functionality and thus, we decided to validate the potential of this combination of factors in an alternative pluripotent stem cell line. We generated Opti-OX hiPSC with the 3TFs or the 3TFs+RORc FoP system, validated the upregulation of these factors after 24 h of dox treatment and then induced differentiation following the protocol established above. FoP-Heps derived from hiPSC also displayed cobblestone morphology (FIG. 5A) and expressed Albumin, AFP and SERPINA1/A1AT at higher levels when RORc was overexpressed (FIGS. 5B, 5C, 5D, and 5E). In addition, the presence of RORc significantly increase basal CYP3A4 activity thereby confirming the positive effect of this factor on functional maturity of FoP-Heps (FIG. 5F). Overall, these results showed that overexpression of specific nuclear receptors was compatible with the generation of FoP-hepatocytes. In particular, overexpression of RORc could improve the functionality of hepatocytes generated by overexpression of 3 LETFs confirming the role of this nuclear receptor in hepatocyte maturation.

### 4TF FoP-Heps Display Functional Characteristics In Vitro

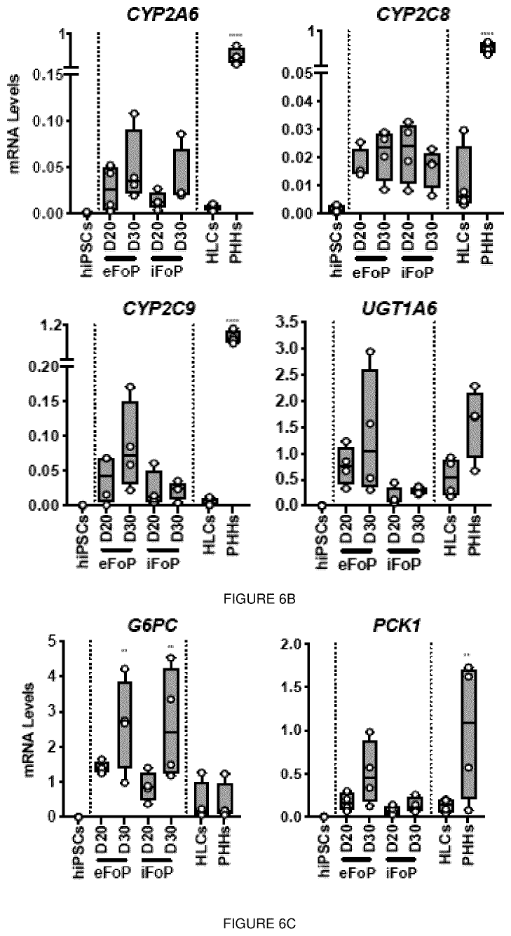

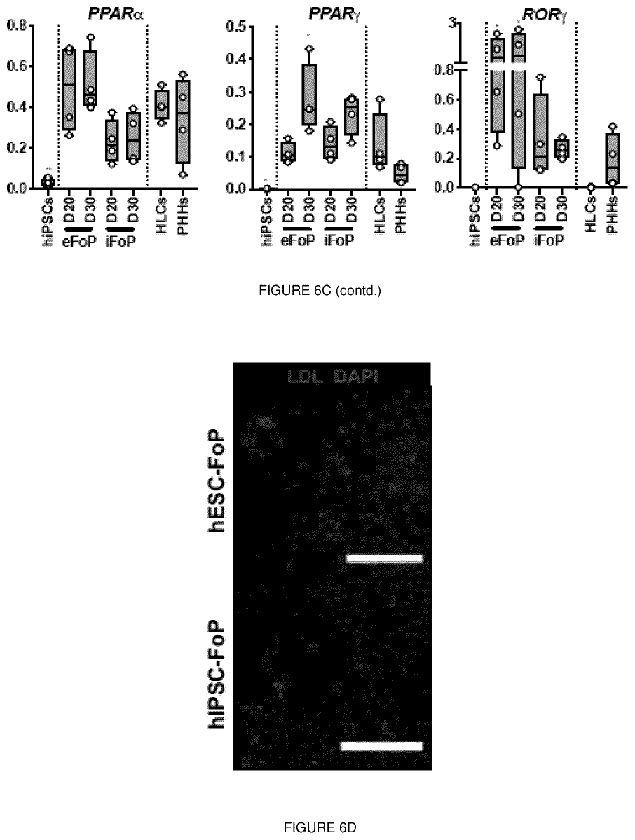

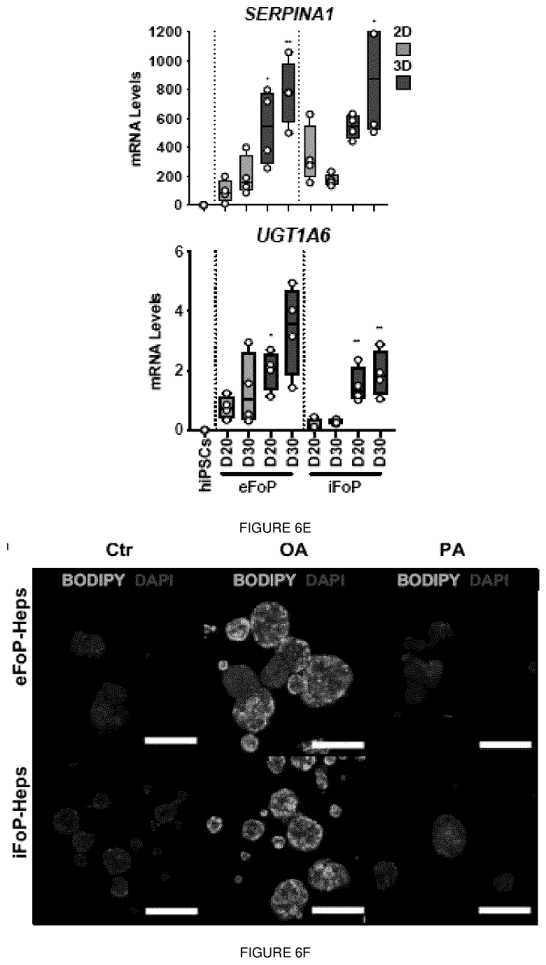

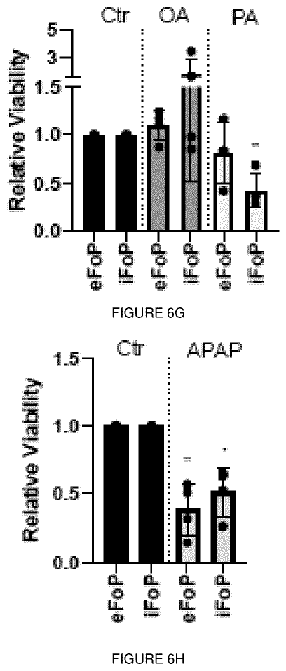

Next, we sought to further characterise the functionality of the 4TF (HNF1A, HNF6, FOXA3 and RORc) FoP-Heps derived from either hESC (eFoP-Heps) or hiPSCs (iFoP-Heps) in comparison with HLCs generated by the direct differentiation and PHHs. CYP3A4 activity was significantly higher in 4TF FoP-Heps forward programmed after 20 days than those achieved by HLCs after 30 days of directed differentiation (FIG. 6A). In addition, we analysed the expression of markers associated with hepatic metabolic functions such as phase I (cytochrome P450 enzymes) and phase II (UGTs) biotransformation, gluconeogenesis (G6PC and PCK1) and lipid (PPARα, PPARy, FASN and APOA1) and bile acid (NR1H4) metabolism. FoP-Heps expressed a range of these functional markers confirming the acquisition of hepatic functionality (FIGS. 6B, and 6C). Overall, the levels of expression achieved by forward programming were equivalent to those achieved by direct differentiation with the exception of gluconeogenesis genes which were increased in FoP-Heps (FIG. 6C). Interestingly, expression of gluconeogenesis and lipid metabolism genes was comparable between FoP-Heps and PHHs for. However, induction of cytochromeP450 genes remains challenging, indicating that the acquisition of this specific hepatic function could need further refinement of our protocol (FIG. 6B). Of note, in FoP-Heps RORc remained expressed at physiological levels at the end of our protocol (FIG. 6B). In addition to expressing mature hepatocyte markers, eFoP-Heps and iFoP-Heps were also able to uptake LDL from the culture medium confirming their capacity to transport lipids (FIG. 6D). In order to further explore their capacity to metabolise lipids, FoP-Heps were grown in 3D for an additional 5 (D20) or 10 (D30) days as we recently observed that such culture conditions facilitate lipid accumulation in HLCs (Carola Morell, personal communication). We first confirmed that FoP-Heps grown in 3D retained the expression of hepatocyte markers (FIG. 6E). Interestingly, SERPINA1 or UGT1A6 expression increased in these conditions suggesting an increase in functional maturation promoted in 3D (FIG. 6E). We then tested the capacity of both eFoP and iFoP-Heps to respond to fatty acids by treating these cells with both oleic acid (OA) and palmitic acid (PA) which are known to induce steatosis and lipotoxicity respectively (Ricchi et al., 2009). In line with their known effect on hepatocytes, OA treatment induced a strong accumulation of lipids as shown by BODIPY staining (FIG. 6F) while PA treatment induced a reduction in cell viability consistent with lipotoxicity (FIG. 6G). Thus, FoP-Heps appear to react to fatty acid similarly to their primary counterpart. Finally, we explored the interest of FoP-Heps for modelling the hepatoxic effect of paracetamol/acetaminophen. For that, Fop-Heps were grown in the presence of an acetaminophen (APAP) dose known to induce liver failure. This treatment resulted in a 50% reduction in cell viability (FIG. 6H) suggesting that FoP-Heps could be used for cytotoxic studies. In summary, these results showed that 4TF FoP-Heps derived from either hESC or hiPSCs display characteristics of functional hepatocytes such as expression of genes involved in drug, lipid, glucose and bile acid metabolism, capacity to uptake LDL and fatty acids from the culture medium, as well as response to hepatotoxic factors, demonstrating their potential interest for modelling liver disorder in vitro and toxicology screening.

### 5TF FoP-Heps Display Functional Characteristics In Vitro

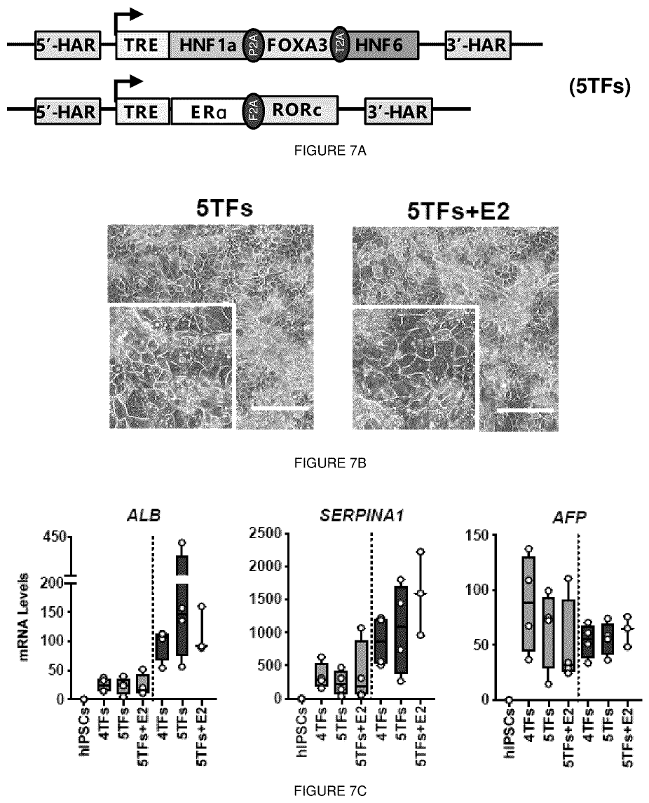

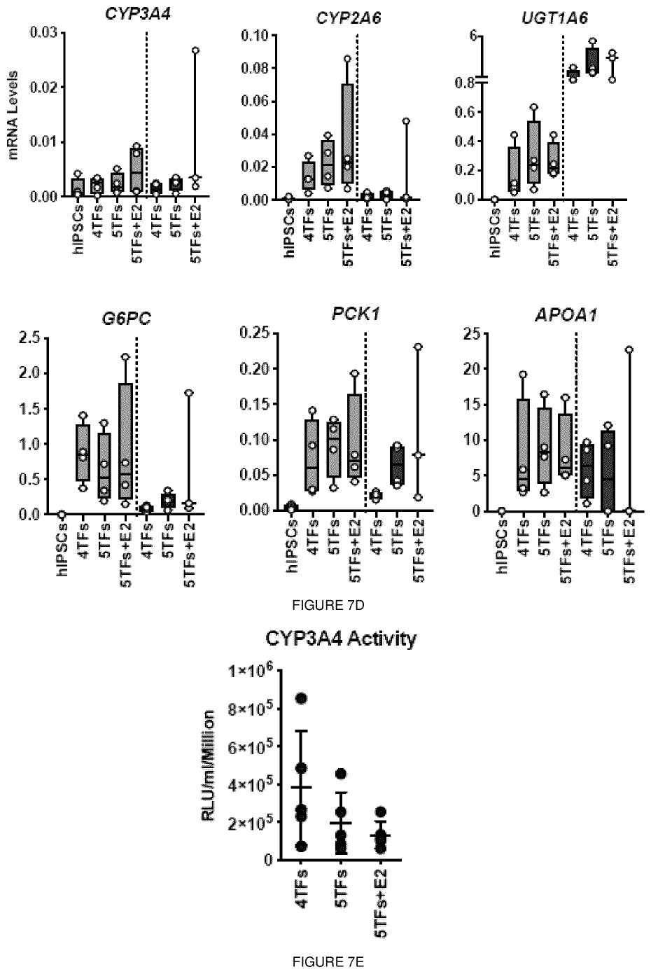

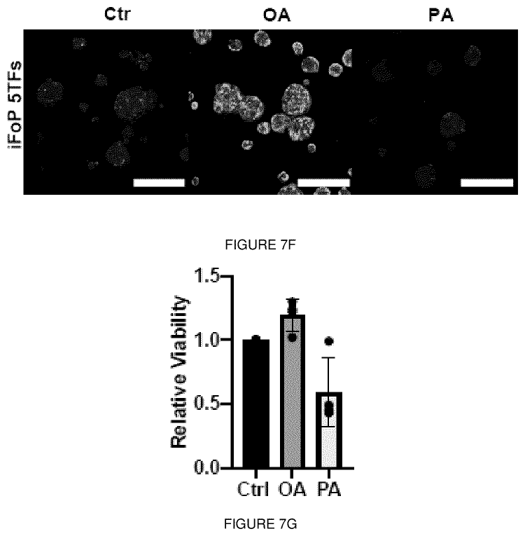

Next, we sought to further characterise the functionality of the 5TF (HNF1A, HNF6, FOXA3, RORc, ERα) FoP-Heps derived from hiPSCs (iFoP-Heps) in comparison with 4TF iFoP-Heps. Overexpression of the 5TFs in the presence or absence of eostrogen allowing the production of cells resembling hepatocytes (FIG. 7B). These cells were shown to express key hepatocyte markers (FIGS. 7C and 7D). iFop-Heps generated with 5 FTs were also found to display a high level of CyP3A4 activity (FIG. 7E), lipid transport/accumulation (FIG. 7F) and lipotoxicity (FIG. 7G).

In this study, we have established a method to forward program hPSCs into hepatocytes. The success of this approach depends on the selection of TFs, combining factors controlling early liver development and regulators of adult hepatic functions. Nonetheless, most forward programming methods rely on a master regulator to convert hPSCs into a specific cell type. As an example, neurons and muscle cells can be generated by the simple overexpression of NGN2 and MYOD respectively (Pawlowski et al., 2017). Our results show that production of hepatocytes requires a more complex process involving 3 transcription factors but also a culture media supporting primary hepatocytes. Furthermore, our best LETFs combination did not include HNF4A, which is known to be a key regulator of hepatocyte function in the adult liver. On the contrary, removing HNF4A significantly improved the identity of the hepatocytes generated. Similar observations were recently reported for the direct reprogramming of human umbilical vein endothelial cells into bipotent hepatocyte progenitor cells where HNF4A was found to be detrimental (Inada et al., 2020). HNF4A is essential not only in adult liver but also during development, especially in the establishment of the liver bud (Gordillo et al., 2015). Thus, HNF4A might have also a role in preserving foetal liver cells such as hepatoblast and, its overexpression during forward programming could block the acquisition of an adult hepatocytic identity. This example illustrates the challenges to identify factors which are uniquely express in the adult liver. Importantly, FoP-Heps generated by LETF overexpression acquired an hepatocytic identity with reduced adult functions, suggesting that this cocktail of transcription might only convert hiPSCs into foetal-like cells. Thus, we decided to add factors which could direct functional maturation of the liver. This latest category of factors were identified by performing a transcriptomic and epigenetic comparison of PHHs and HLC generated by direct differentiation. The focus on HLCs was based on their well characterised foetal state and also the broad experience with the cells. These analyses identified a subset of nuclear receptors that were exclusively expressed in PHHs and in the adult liver thereby confirming the relevance of our approach. Of particular interest, RORc, ERα and AR were identified as key candidate for controlling functional maturation in hepatocytes. Importantly, nuclear receptors are well known to control diverse liver functions including lipid and glucose homeostasis, bile acid clearance, xenobiotic sensing and regeneration (Rudraiah et al., 2016). Both steroid hormonal receptors ERE and AR have been shown to have roles in the regulation of energy homeostasis in the liver (Shen & Shi, 2015). Moreover, ERE is involved in cholesterol clearance (Zhu et al., 2018) and has also been associated with liver regeneration (Kao et al., 2018) and bilirubin metabolism through CYP2A6 (Kao et al., 2017). RORc is a nuclear receptor expressed in peripheral tissues including liver, muscle and adipose tissue and has been proposed to function as an intermediary between the circadian clock and glucose/lipid metabolism (Cook et al., 2015). Moreover, RORy-deficient mice exhibit insulin sensitivity and reduced expression of gluconeogenesis, lipid metabolic markers, and a subset of phase I enzymes involved in bile acid synthesis and phase II enzymes (Kang et al., 2007; Takeda, Kang, Freudenberg, et al., 2014; Takeda, Kang, Lih, et al., 2014). Based on these previous reports, we propose that the overexpression of RORc and other nuclear receptors could improve specific functions in FoP-Heps by activating a subset of target genes in the hepatic context induced by the LETFs overexpression. Importantly, hepatocyte functionality is spatially different across the liver lobule, being influenced by the gradient of oxygen, nutrients and signalling (Trefts et al., 2017). This hepatic zonation drives different metabolic processes in regard to glucose, lipids, iron, or even xenobiotics, which are under the control of different transcriptomic programs (Halpern et al., 2017). Thus, we expect that different combinations of nuclear factors in the background induced by LETFs overexpression could enable the production of hepatocytes with a distinct repertoire of functions.

FoP-Heps generated with the overexpression of the 4TFs (HNF1A, HNF6, FOXA3 and RORc) displayed functional features of adult hepatocytes including Albumin and A1AT secretion, basal CYP3A4 activity, expression of Phase I/Phase II enzymes, gluconeogenesis and lipid metabolism markers, capacity to uptake LDL and fatty acids as well as response to toxic compounds. Nonetheless, CYP3A4 expression remains limited and this gene remains difficult to induce in vitro. Thus, additional TFs could be necessary to generate FoP-Heps exhibiting the full spectrum of functional activities displayed by PHHs. Similarly, culture conditions could be further improved to support key hepatic functions. Indeed, the basal medium used in our protocol does not prevent dedifferentiation of PHHs and thus might not be compatible with the production of fully functional cells by forward programming. Nonetheless, the forward programming method established here presents several advantages over conventional directed differentiation protocols. This is a robust two-steps method which bypasses the need for multi-step differentiations which are often associated with batch-to-batch variability. Furthermore, forward programming is faster, generating functional cells in 20 days, as opposed to 30-35 days for direct differentiation. Finally, the yield of cells seems favourable and compatible with large-scale production. Indeed, we observed that forward programming was associated with an 6-8 fold increase in cell number during differentiation while the yield of direct differentiation is lower. Taken together, our results describe the first method for generating hepatocytes using forward programming. This approach represents the first step towards the high-throughput and large-scale production of specialized hepatocytes displaying a spectrum of functions relevant for different applications in disease modelling and drug screening.

### SEQUENCES

TABLE 1
Sequences of primers used for cloning.

| Gene | Primer | Sequence 5'-3' (GSG linker sequence underlined) |
|---|---|---|
| HNF1A | Start_F | CAC TTT TGT CTT ATA CTT ACT AGT GCC ACC ATG GTT TCT AAA CTG AGC CAG |
| | | CTG CAG |
| HNF1A | P2A_R | TTC CAC GTC TCC TGC TTG CTT TAA CAG AGA GAA GTT CGT GGC TCC GGA GCC |
| | | CTG GGA GGA AGA GGC CAT CTG G |
| HNF4A | E2A_F | TAT GCT CTC TTG AAA TTG GCT GGA GAT GTT GAG AGC AAC CCT GGA CCT GTC |
| | | AGC GTG AAC GCG CCC CT |
| HNF4A | Stop_R | AGA GGA TCC CCG GGT ACC GAG CTC GAA TTC CTA GAT AAC TTC CTG CTT GGT |
| | | GAT GGT CG |
| HNF4A | P2A_F | TCT CTG TTA AAG CAA GCA GGA GAC GTG GAA GAA AAC CCC GGT CCT GTC AGC |
| | | GTG AAC GCG CCC CT |
| HNF4A | T2A_R | CTC CTC CAC GTC ACC GCA TGT TAG AAG ACT TCC TCT GCC CTC TCC GGA GCC |
| | | GAT AAC TTC CTG CTT GGT GAT GGT CG |
| HNF4A | Start_F | CAC TTT TGT CTT ATA CTT ACT AGT GCC ACC ATG GTC AGC GTG AAC GCG CCC |
| HNF4A | P2A_R | TTC CAC GTC TCC TGC TTG CTT TAA CAG AGA GAA GTT CGT GGC TCC GGA GCC |
| | | GAT AAC TTC CTG CTT GGT GAT GGT CG |
| FOXA3 | T2A_F | AGT CTT CTA ACA TGC GGT GAC GTG GAG GAG AAT CCC GGC CCT CTG GGC TCA |
| | | GTG AAG ATG GAG GC |
| FOXA3 | E2A_R | CTC AAC ATC TCC AGC CAA TTT CAA GAG AGC ATA ATT AGT ACA CTG TCC GGA |
| | | GCC GGA TGC ATT AAG CAA AGA GCG GGA ATA G |
| FOXA3 | P2A_F | TCT CTG TTA AAG CAA GCA GGA GAC GTG GAA GAA AAC CCC GGT CCT CTG GGC |
| | | TCA GTG AAG ATG GAG GC |
| FOXA3 | T2A_R | CTC CTC CAC GTC ACC GCA TGT TAG AAG ACT TCC TCT GCC CTC TCC GGA GCC |
| | | GGA TGC ATT AAG CAA AGA GCG GGA ATA G |
| FOXA3 | T2A_F | AGT CTT CTA ACA TGC GGT GAC GTG GAG GAG AAT CCC GGC CCT CTG GGC TCA |
| | | GTG AAG ATG GAG GC |
| FOXA3 | Stop_R | AGA GGA TCC CCG GGT ACC GAG CTC GAA TTC CTA GGA TGC ATT AAG CAA AGA |
| | | GCG GGA ATA G |
| HNF6 | P2A_F | TCT CTG TTA AAG CAA GCA GGA GAC GTG GAA GAA AAC CCC GGT CCT AAC GCG |
| | | CAG CTG ACC ATG GAA GC |
| HNF6 | T2A_R | CTC CTC CAC GTC ACC GCA TGT TAG AAG ACT TCC TCT GCC CTC TCC GGA GCC |
| | | TGC TTT GGT ACA AGT GCT TGA TGA AGA AGA T |
| HNF6 | T2A_F | AGT CTT CTA ACA TGC GGT GAC GTG GAG GAG AAT CCC GGC CCT AAC GCG CAG |
| | | CTG ACC ATG GAA GC |
| HNF6 | Stop_R | AGA GGA TCC CCG GGT ACC GAG CTC GAA TTC CTA TGC TTT GGT ACA AGT GCT |
| | | TGA TGA AGA AGA T |
| RORy | E2A_F | TAT GCT CTC TTG AAA TTG GCT GGA GAT GTT GAG AGC AAC CCT GGA CCT GAC |
| | | AGG GCC CCA CAG AGA CAG |
| RORy | Stop_R | AGA GGA TCC CCG GGT ACC GAG CTC GAA TTC CTA CTT GGA CAG CCC CAC AGG |
| | | TGA C |
| ESR1 | E2A_F | TAT GCT CTC TTG AAA TTG GCT GGA GAT GTT GAG AGC AAC CCT GGA CCT ACC |
| | | ATG ACC CTC CAC ACC AAA GCA |
| ESR1 | Stop_R | AGA GGA TCC CCG GGT ACC GAG CTC GAA TTC CTA GAC CGT GGC AGG GAA ACC |
| | | CTC |
| AR | E2A_F | TAT GCT CTC TTG AAA TTG GCT GGA GAT GTT GAG AGC AAC CCT GGA CCT GAA |
| | | GTG CAG TTA GGG CTG GGA AG |
| AR | Stop_R | AGA GGA TCC CCG GGT ACC GAG CTC GAA TTC CTA CTG GGT GTG GAA ATA GAT |
| | | GGG CTT G |

TABLE 2
Sequences of primers used for qPCR

| Gene | Forward | Reverse |
|---|---|---|
| AFP | TGCGGCCTCTTCCAGAAACT | TAATGTCAGCCGCTCCCTCG |
| ALB | CCTTTGGCACAATGAAGTGGGTAACC | CAGCAGTCAGCCATTTCACCATAG |
| APOA1 | AGACAGCGGCAGAGACTATG | CCAGTTGTCAAGGAGCTTTAGG |
| CYP2A6 | CAGCACTTCCTGAATGAG | AGGTGACTGGGAGGACTTGAGGC |
| CYP2C8 | CATTACTGACTTCCGTGCTACAT | CTCCTGCACAAATTCGTTTTCC |
| CYP2C9 | GCCGGCATGGAGCTGTTTTTAT | GCCAGGCCATCTGCTCTTCTT |
| CYP3A4 | TGTGCCTGAGAACACCAGAG | GTGGTGGAAATAGTCCCGTG |
| FASN | GCAAGCTGAAGGACCTGTCT | AATCTGGGTTGATGCCTCCG |
| FOXA3 | TGGGCTCAGTGAAGATGGAG | GGGGATAGGGAGAGCTTAGAG |
| G6PC | GTGTCCGTGATCGCAGACC | GACGAGGTTGAGCCAGTCTC |
| HHEX | GCCCTTTTACATCGAGGACA | AGGGCGAACATTGAGAGCTA |
| HNF1A | TGGCCATGGACACGTACAG | GCTGCTTGAGGGTACTTCTG |
| HNF4A | CATGGCCAAGATTGACAACCT | TTCCCATATGTTCCTGCATCAG |
| HNF6 | GTGTTGCCTCTATCCTTCCCAT | CGCTCCGCTTAGCAGCAT |
| NANOG | CATGAGTGTGGATCCAGCTTG | CCTGAATAAGCAGATCCATGG |
| NR1H4 | ACTGAACTCACCCCAGATCAA | TGGTTGCCATTTCCGTCAAA |
| PBGD | GGAGCCATGTCTGGTAACGG | CCACGCGAATCACTCTCATCT |
| PCK1 | ACACAGTGCCCATCCCCAAA | GGTGCGACCTTTCATGCACC |
| POU5F1 | AGTGAGAGGCAACCTGGAGA | ACACTCGGACCACATCCTTC |
| PPARa | CCCTCCTCGGTGACTTATCC | CGGTCGCACTTGTCATACAC |
| PPARy | GAGCCTGCATCTCCACCTTAT | AGAAACCCTTGCATCCTTCACA |
| RORy | CTACGGCAGCCCCAGTTT | GCTGGCATGTCTCCCTGTA |
| RPLP0 | GGCGTCCTCGTGGAAGTGAC | GCCTTGCGCATCATGGTGTT |
| SERPINA1 | CCACCGCCATCTTCTTCCTGCCTGA | GAGCTTCAGGGGTGCCTCCTCTG |
| SOX17 | CGCACGGAATTTGAACAGTA | GGA TCAGGGACCTGTCACAC |
| TBX3 | TGGAGCCCGAAGAAGAGGTG | TTCGCCTTCCCGACTTGGTA |
| UGT1A1 | TGATCCCAGTGGATGGCAGC | CAACGAGGCGTCAGGTGCTA |
| UGT1A6 | GGAGCCCTGTGATTTGGAGAGT | GACCCCGGTCACTGAGAACC |

### REFERENCES

Baxter et al (2015) J Hepatol 62(3) 581-589.
Bertero, A. et al (2016, Dec. 1). Development, 143(23), 4405-4418.
Boon, R et al. (2020, Mar. 13). Nat Commun, 11(1), 1393.
Brown, S. et al Stem Cells, 29(8), 1176-1185.
Chen, G. et al (2011, May) Nat Methods, 8(5), 424-429.
Consortium, E. P. (2012, Sep. 6). Nature, 489(7414), 57-74.
Cook, D. N., et al. (2015). Nucl Receptor Res, 2. 101185
Creyghton, M. P. et al (2010, Dec. 14). Proc Natl Acad Sci USA, 107(50) 21931-21936
Dhawan, A. et al (2020, May) J Hepatol, 72(5), 877-884
Du, Y. et al (2014 Mar. 6). Cell Stem Cell 14(3) 394-403.
Gordillo, M et al (2015, Jun. 15). Development, 142(12), 2094-2108.
Grandy, R. et al (2019, Jan. 24). Annu Rev Pathol 14, 449-468.
Halpern, K. B. et al (2017, Feb. 16). Nature, 542(7641), 352-356
Hannan, N. R. et al (2013, February) Nat Protoc, 8(2), 430-437.
Hay et al Stem Cells (2008) April; 26(4):894-902
Heinz, S., et al. (2010, May 28). Mol Cell 38(4) 576-589
Huang, P. et al (2014, Mar. 6). Cell Stem Cell 14(3) 370-384.
Inada, H., et al (2020, Oct. 21). Nat Commun, 11(1) 5292.
Kang, H. S. et al (2007, Oct. 22). Physiol Genomics 31(2) 281-294.
Kao, T. L., et al (2017, November). Cell Transplant 26(11) 1822-1829.
Kao, T. L. et al (2018). Theranostics, 8(10), 2672-2682.
Lambert, S. A. et al (2018, Oct. 4). Cell, 175(2), 598-599.
Lau, H. H et al (2018, May). J Hepatol, 68(5), 1033-1048.
Love, M. I. et al (2014) Genome Biol 15(12) 550.
Mitry, R. R. et al (2002, December). Semin Cell Dev Biol, 13(6), 463-467.
Nakamori, D. et al (2016, Jan. 15). Biochem Biophys Res Commun, 469(3), 424-429.
Palakkan, A. A. et al (2017, April). Biomed Rep, 6(4), 367-373.
Patro, R., et al (2017, April). Nat Methods, 14(4), 417-419
Pawlowski, M. et al (2017, Apr. 11). Stem Cell Reports 8(4) 803-812.
Rashid, S. T. et al (2010, Sep. 1). J Clin Invest, 120(9), 3127-3136.
Ricchi, M. et al (2009, May) J Gastroenterol Hepatol 24(5) 830-840.
Rombaut, M. et al (2021, May 11). J Hepatol. 75 690-705
Rudraiah, S. et al (2016). Annu Rev Pharmacol Toxicol, 56, 605-626.
Schrem, H. et al (2002, March). Pharmacol Rev, 54(1), 129-158.
Segeritz, C. P. et al (2018, October). J Hepatol, 69(4), 851-860.
Shen, M., & Shi, H. (2015). Int J Endocrinol, 2015, 294278.
Silier et al Stem Cell Reports 2015 4 5 939-952
Si-Tayeb, K. et al (2010, Feb. 16) Dev Cell, 18(2) 175-189.
Szkolnicka, D., & Hay, D. C. (2016, June). Stem Cells, 813 34(6), 1421-1426.
Takeda, Y., et al (2014) PLoS Genet,
Takeda, Y. et al (2014). Nucleic Acids Res, 42(16), 10448-
Touboul, T. et al (2010, May) Hepatology,
Trefts, E. et al (2017, Nov. 6). Curr Biol, 27(21),
Wang, A. et al (2015, April) Cell Stem Cell, 16(4), 386-399.
Yiangou, L. et al (2018, Apr. 5). Cell Stem Cell, 22(4), 485-499.
Yu, G., & He, Q. Y. (2016, February). Mol Biosyst, 12(2), 477-479.
Yu, G. et al (2012, May) OMICS, 16(5), 284-287.
Yusa, K. et al (2011, Oct. 20). Nature, 478(7369), 391-394.
Zhao, D. et al (2013, January). Cell Res, 23(1), 157-161.
Zhu, L. et al (2018, February). Mol Metab, 8, 106-116.

1. A method of producing hepatocytes comprising;
(i) providing a population of iPSCs
(ii) introducing a set of transcription factors consisting of (i) HNF1A; HNF6; FOXA3; and RORc; or (ii) HNF1A; HNF6; FOXA3; RORc; and ERα into the population of iPSCs, and
(iii) culturing the population, such that one or more cells in the population are programmed into hepatocytes.

2. The method according to claim 1 wherein the set of transcription factors consists of HNF1A; HNF6; FOXA3; RORc and ERα.

3. The method according to claim 1 wherein the set of transcription factors consists of HNF1A; HNF6; FOXA3; and RORc.

4. The method according to claim 1, wherein the hepatocytes are functionally mature.

5. The method according to claim 1, wherein the pluripotent stem cells are human pluripotent stem cells (hPSCs).

6. The method according to claim 1, wherein the pluripotent stem cells are induced pluripotent stem cells (iPSCs).

7. The method according to claim 1 wherein the transcription factors are human transcription factors.

8. The method according to claim 1, wherein the population of cells is cultured under suitable conditions and for a sufficient period of time to allow one or more cells to display a hepatocyte phenotype.

9. The method according to claim 1, wherein during step (iii) the cells said population do not display endoderm and hepatic progenitor phenotypes.

10. The method according to claim 1, wherein the set of transcription factors is introduced into the PSCs by:
(a) expressing a nucleic acid encoding the combination of transcription factors in the PSCs;
(b) contacting the PSCs with the combination of transcription factor mRNAs or proteins; or
(c) activating endogenous expression of genes encoding the combination of transcription factors in the PSCs.

11. The method according to claim 10, wherein the nucleic acid encoding the set of transcription factors is expressed in the PSCs by transfecting the PSCs with one or more vectors comprising said nucleic acids operably linked to one or more regulatory elements for expression of the nucleic acids in the PSCs.

12. The method according to claim 11, wherein the one or more regulatory elements are inducible regulatory elements.

13. The method according to claim 11, wherein the vectors are lentiviral vectors.

14. The method according to claim 1, wherein the population of PSCs is cultured for at least 7 days.

15. The method according to claim 1, wherein at least 5% of said population are forward programmed into hepatocytes following step (iii).

16. The method according to claim 1, comprising isolating and/or purifying hepatocytes.

17. The method according to claim 1, comprising expanding the population of hepatocytes.

18. The method according to claim 1, comprising storing the population of hepatocytes.

19. A population of hepatocytes produced by the method according to claim 1.

20. A pharmaceutical composition comprising the population of hepatocytes according to claim 19, and a pharmaceutically acceptable excipient.

21. A method of treating a disease in a subject in need thereof, the method comprising administrating the population of hepatocytes according to claim 19 to the subject.

22. (canceled)

23. A method of treating a liver disorder comprising;
administering the population of hepatocytes according to claim 19 to an individual in need thereof.

24-25. (canceled)

26. A method of screening for a compound useful in the treatment of a liver disorder comprising;
contacting the population of hepatocytes according to claim 19 with a test compound, and;
determining the effect of the test compound on said hepatocytes or the effect of the hepatocytes on the test compound.

27. A method of determining the hepatotoxicity of a compound comprising:
contacting the isolated hepatocyte cells according to claim 19 with a test compound, and
determining the effect of the test compound on said hepatocytes.
